SEQUENCE LISTING (10 sequences)

SEQ ID NO: 1 - protein, 121 aa, synthetic construct
  REGION 1..121: note = recombinant peptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 111 aa, synthetic construct
  REGION 1..111: note = recombinant peptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 3 - protein, 451 aa, synthetic construct
  REGION 1..451: note = recombinant peptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 218 aa, synthetic construct
  REGION 1..218: note = recombinant peptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, synthetic construct
  REGION 1..5: note = recombinant peptide
SSWMN

SEQ ID NO: 6 - protein, 17 aa, synthetic construct
  REGION 1..17: note = recombinant peptide
RIYPGDGDTNYNVKFKG

SEQ ID NO: 7 - protein, 12 aa, synthetic construct
  REGION 1..12: note = recombinant peptide
SGFITTVRDFDY

SEQ ID NO: 8 - protein, 15 aa, synthetic construct
  REGION 1..15: note = recombinant peptide
RASESVDTFGISFMN

SEQ ID NO: 9 - protein, 7 aa, synthetic construct
  REGION 1..7: note = recombinant peptide
EASNQGS

SEQ ID NO: 10 - protein, 9 aa, synthetic construct
  REGION 1..9: note = recombinant peptide
QQSKEVPFT

(19) United States
(12) Patent Application Publication
(10) Pub. No.: US 2023/0287114 A1
(43) Pub. Date: Sep. 14, 2023

(54) USE OF AN ANTI-CD19 ANTIBODY TO TREAT AUTOIMMUNE DISEASE

(71) Applicant: Viela Bio, Inc., Deerfield, IL (US)

(72) Inventors: Dewei SHE, McLean, VA (US); John Nolan RATCHFORD, Lutherville, MD (US); Eliezer KATZ, Durham, NC (US); William A. REES, North Bethesda, MD (US)

(21) Appl. No.: 18/303,303

(22) Filed: Apr. 19, 2023

Related U.S. Application Data
(63) Continuation of application No. PCT/US2021/057443, filed on Oct. 29, 2021.
(60) Provisional application No. 63/107,182, filed on Oct. 29, 2020.
(60) Provisional application No. 63/143,541, filed on Jan. 29, 2021.
(60) Provisional application No. 63/178,286, filed on Apr. 22, 2021.

(51) Int. Cl. C07K 16/28; A61K 9/00; A61K 31/573; A61P 37/06

(52) U.S. Cl. CPC C07K 16/2803; A61K 9/0019; A61K 31/573; A61P 37/06; C07K 2317/24

(57) ABSTRACT

Methods for using an anti-CD19 antibody to treat autoimmune disease are disclosed herein. In particular the use of VIB551, a humanised, affinity-optimised, afucosylated IgG1 kappa monoclonal antibody to treat Neuromyelitis optica spectrum disorder.

### CROSS-REFERENCE

This application is a continuation of International Patent Application No. PCT/US2021/057443, filed Oct. 29, 2021, which claims the benefit of U.S. Provisional Application Nos. 63/107,182, filed Oct. 29, 2020, 63/143,541, filed Jan. 29, 2021, and 63/178,286, filed Apr. 22, 2021, the contents of each of which are incorporated herein by reference.

### REFERENCE TO SEQUENCE LISTING

The contents of the electronic sequence listing (HOPA_030_03US_SeqList_ST26.xml; Size: 14,374 bytes; and Date of Creation: Mar. 15, 2023) are herein incorporated by reference in its entirety.

### BACKGROUND

Neuromyelitis optica spectrum disorder (NMOSD) is a severe, autoimmune, inflammatory, central nervous system disease with a prevalence of 0-5-4-4/100 000. (Cree B A, Bennett J L, Sheehan M, et al. Placebo-controlled study in neuromyelitis optica—ethical and design considerations. Mult Scler 2016; 22: 862-72.) NMOSD presents with optic neuritis, transverse myelitis, and, less commonly, diencephalic, brainstem, and cerebral hemisphere attacks. (Cree B A, Bennett J L, Sheehan M, et al Placebo-controlled study in neuromyelitis optica—ethical and design considerations. Mult Scler 2016; 22: 862-72.) Incomplete recovery from attacks is typical, and patients are at risk of death from respiratory failure. (Wingerchuk D M, Lennon V A, Lucchinetti C F, Pittock S J, Weinshenker B G. The spectrum of neuromyelitis optica. Lancet Neurol 2007; 6: 805-15.)

Once considered a multiple sclerosis variant, NMOSD is now recognised as a distinct disease, characterised by astroglial injury, demvelination, and significant neuronal loss; most injury occurs during acute attacks (Wingerchuk D M, Lennon V A, Lucchinetti C F, Pittock S J, Weinshenker B G. The spectrum of neuromyelitis optica Lancet Neurol 2007: 6: 805-15; Fujihara K. Misu T, Nakashima I, et al. Neuromyelitis optica should be classified as an astrocytopathic disease rather than a demyelinating disease. Chin Exp Neuroimmunol 2012: 3; 58-73.). Highly specific serum autoantibodies against the astrocyte water channel aquaporin-4 (AQP4)-immunoglobulin G (IgG) are detected in 60-80% of patients and are likely pathogenic (Weinshenker B G. Wingerchuk D M, Vukusic S. et al. Neuromyelitis optica IgG predicts relapse after longitudinally extensive transverse myelitis. Ann Neurol 2006; 59: 566-9; Bennett J L, Lam C, Kalluri S R, et al. Intrathecal pathogenic anti-aquaporin-4 antibodies in early neuromyelitis optica. Ann Neurol 2009; 66: 617-29; Jarius S. Frederikson J, Waters P, et al. Frequency and prognostic impact of antibodies to aquaporin-4 in patients with optic neuritis. J Neurol Sci 2010: 298: 158-62; Saadoun S, Waters P, Bell B A, Vincent A, Verkman A S, Papadopoulos M C. Intra-cerebral injection of neuromyelitis optica immunoglobulin G and human complement produces neuromyelitis optica lesions in mice. Brain 2010; 133: 349-61). In the presence of complement or inflammatory T-cell response, AQP4-IgG causes disease-specific central nervous system injury. (Bennett J L. Lam C, Kalluri S R, et al. Intrathecal pathogenic anti-aquaporin-4 antibodies in early neuromyelitis optica Ann Neurol 2009; 66: 617-29.) Multiple lines of evidence suggest that NMOSD is predominantly a B-cell-mediated disorder resulting from pathologic autoantibody production, pro-inflammatory cytokine secretion, and B-cell antigen presentation. (Bennett J L, O'Connor K C, Bar-Or A, et al. B lymphocytes in neuromyelitis optica. Neurol Neuroimmunol Neuroinflamm 2015; 2: e104.)

VIB551 is a humanised, affinity-optimised, afucosylated IgG1 kappa monoclonal antibody that binds to the B-cell surface antigen CD19. In contrast to anti-CD20 monoclonal antibodies that recognise and deplete a subset of CD20-expressing T lymphocytes (in addition to B lymphocytes) (Palanichamy A, Jahn S, Nickles D, et al. Rituximab efficiently depletes increased CD20-expressing T cells in multiple sclerosis patients. J Immunol 2014; 193: 580-6), anti-CD19 antibodies recognise and deplete lymphocytes exclusively from the B-cell lineage.

### BRIEF SUMMARY

The description provides for a method of treating neuromyelitis optica spectrum disorder (NMOSD), the method comprising: administering the anti-CD19 antibody VIB551 to a patient in need of treatment for NMOSD, wherein the patient had been previously treated with an anti-CD20 antibody, and wherein the patient had an NMOSD attack while being treated with the anti-CD20 antibody. The description provides for a method of treating NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects. VIB551 is administered intravenously at a dose of 300 mg every 6 months.

The description provides for a method of treating neuromyelitis optica spectrum disorder (NMOSD), the method comprising: administering the anti-CD19 antibody VIB551 to a patient in need of treatment for NMOSD, wherein the patient had been previously treated with an anti-CD20 antibody, and wherein the patient has not been treated with anti-CD20 for up to 6 months or up to 12 months prior to administering the anti-CD19 antibody VIB551, optionally, wherein the patient had an NMOSD attack while being treated with the anti-CD20 antibody. The description provides for a method of treating NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects, VIB551 is administered intravenously at a dose of 30) mg every 6 months.

The description provides for a method of treating NMOSD, the method comprising: administering the anti-CD19 antibody VIB551 to a patient in need of treatment for NMOSD, wherein the patient is an astrocyte water channel aquaporin-4 (AQP4)-IgG+ patient, wherein the VIB551 is administered intravenously at a dose of 300 mg every 6 months, wherein the patient had been previously treated with an anti-CD20 antibody, and wherein the patient had an NMOSD attack while being treated with the anti-CD20 antibody. The description provides for a method of treating NMOSD, the method comprising: administering the anti-CD19 antibody VIB551 to a patient in need of treatment for NMOSD, wherein the patient is an AQP4-IgG+ patient, wherein the VIB551 is administered intravenously at a dose of 300 mg every 6 months, wherein the patient had been previously treated with an anti-CD20 antibody; and, wherein the patient had an NMOSD attack within 6 months of the last dose of the anti-CD20 antibody.

In aspects, the patient receives at least one initial dose of V1B551. In aspects, VIB551 is administered intravenously with a first initial dose of 300 mg, a second initial dose of 300 mg two weeks after the first initial dose, and subsequent doses of 300 mg every 6 months following the first initial dose. In aspects, oral corticosteroids are co-administered to the patient with the initial VIB551 dose. In particular aspects, oral corticosteroids are administered daily for at least 2 weeks. In one aspect, the anti-CD20 antibody is rituximab.

In aspects, the treating is a reduction in worsening of Kurtzke Expanded Disability Severity Scale (EDSS) in the patient. In aspects, reduction in worsening of EDSS in the patient is: a worsening of fewer than 2 points in EDSS score if the patient has a baseline score of 0; a worsening of fewer than 1 point if the patient has a baseline score of 1 to 5; or a worsening of less than 0.5 point if the patient has a baseline score of 5.5 or more.

In aspects, the treating is a reduction in number of active magnetic resonance imaging (MRI) lesions. In aspects, the active MRI lesions are enlarging T2 MRI lesions. In particular aspects, the treating is a reduction in number of new MRI lesions.

In aspects, the treating is a reduction in worsening of modified Rankin Score in the patient. In particular aspects, the treating is a reduction in frequency of in-patient hospitalizations of the patient related to NMOSD. In aspects, the treating is a reduction of risk of an NMOSD-related attack of the patient. In one aspect, the NMOSD-related attack is characterized by appearance of a new symptom or worsening of an existing symptom related to NMOSD. In a particular aspect, the symptom is an eye symptom. In aspects, the eye symptom is eye pain, blurred vision, loss of vision, or appearance of an optic nerve lesion detected by MRI. In aspects, the symptom is a spinal cord symptom. In one aspect, the spinal cord symptom is deep or radicular pain, extremity paraesthesia, weakness, sphincter dysfunction. Lhermitte's sign, or a spinal cord lesion detectable by MRI. In aspects, the symptom is a brain or brain stem symptom. In aspects, the brain or brainstem symptom is nausea, double vision, oculomotor palsy, vertigo, intractable vomiting, intractable hiccups, dysarthria, dysphagia, weakness, encephalopathy, hypothalamic dysfunction, or a brain or brain stem lesion detectable by MRI. In aspects, the reduction of risk of the NMOSD-related attack is between 60 and 85%. In one aspect, the treating is a reduction in optic neuritis. In a particular aspect, the treating is a reduction of severity of NMOSD-related attacks. In aspects, the reduction of severity of NMOSD-related attacks is the reduction in NMOSD-related attacks graded as major. In aspects, the reduction of severity of NMOSD-related attacks is the reduction in NMOSD attacks requiring in-patient hospitalization. In a particular aspect, the treating is a decrease in NMOSD-related pain in the patient. In aspects, the decrease in NMOSD-related pain is determined by measuring pain in legs of the patient.

In aspects, two weeks prior to the first administering the 300 mg VIB551 every 6 months, an initial 300 mg VIB551 dose is administered to the subject. In particular aspects, oral corticosteroids are co-administered to the patient with the initial 300 mg VIB551 dose. In aspects, the patient is AQP4-IgG seropositive.

The description provides for a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. The description provides for a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects, the VIB551 is administered intravenously at a dose of 300 mg every 6 months.

The description provides for a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody; and, wherein the VIB551 is administered intravenously at a dose of 300 mg every 6 months. The description provides for a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody; and, wherein the VIB551 is administered intravenously at a dose of 300 mg every 6 months.

The description provides for a method of reducing AQP4-IgG titers in a AQP4-IgG+ patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. The description provides for a method of reducing AQP4-IgG titers in a AQP4-IgG+ patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects, the VIB551 is administered intravenously at a dose of 300 mg every 6 months.

The description provides for a method of reducing AQP4-IgG titers in a AQP4-IgG+ patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody; and, wherein the VIB551 is administered intravenously at a dose of 300 mg every 6 months. The description provides for a method of reducing AQP4-IgG titers in a AQP4-IgG+ patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody; and, wherein the VIB551 is administered intravenously at a dose of 300 mg every 6 months.

In aspects, the administration depletes at least 90% of circulating CD20+ B cells for at least six months. In aspects, the administration does not increase risk of infections in the patient. In particular aspects, the VIB551 depletes peripheral blood CD20− plasmablasts and plasma cells within 8 days following the administering.

The description provides for a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. The description provides for a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects, the VIB551 is administered intravenously at a dose of 300 mg every 6 months.

The description provides for a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody; and, wherein the VIB551 is administered intravenously at a dose of 30) mg every 6 months. The description provides for a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody; and, wherein the VIB551 is administered intravenously at a dose of 300 mg every 6 months.

In aspects, the reducing the NMOSD-related disability in the patient is a reduction in a rate of worsening of NMOSD-related disability in the patient. In aspects, the reducing the NMOSD-related disability in the patient is a lessening of NMOSD-related disability in the patient. In one aspect, the NMOSD-related disability is neurological disability. In aspects, the reducing the NMOSD-related disability is determined using EDSS.

The description provides for a method of reducing NMOSD-related attacks in a patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. The description provides for a method of reducing NMOSD-related attacks in a patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. In aspects, the VIB551 is administered intravenously at a dose of 300 mg every 6 months.

The description provides for a method of reducing NMOSD-related attacks in a patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody; and, wherein the VIB551 is administered intravenously at a dose of 300 mg every 6 months. The description provides for a method of reducing NMOSD-related attacks in a patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody; and, wherein the VIB551 is administered intravenously at a dose of 300 mg every 6 months.

In aspects, the NMOSD-related attacks suffered by the patient comprise any one or more of an optic neuritis, a myelitis, or a brainstem attack. In one aspect, the NMOSD-related attacks suffered by the patient are clinically asymptomatic.

In aspects, the patient receives at least one initial dose of VIB551. In aspects, the VIB551 is administered intravenously with a first initial dose of 300 mg, a second initial dose of 300 mg two weeks after the first initial dose, and subsequent doses of 300 mg every 6 months following the first initial dose. In aspects, oral corticosteroids are co-administered to the patient with the initial 30) mg VIB551 dose. In aspects, the oral corticosteroids are administered daily for at least 2 weeks. In aspects, the anti-CD20 antibody is rituximab.

In aspects, the VIB551 comprises a heavy chain variable region (VH) comprising the amino acid of SEQ ID NO:1 and a light chain variable region (VL) comprising the amino acid of SEQ ID NO: 2.

The description provides for a method of treating NMOSD, the method comprising: administering an anti-CD19 antibody to a patient in need of treatment for NMOSD, wherein the patient had been previously treated with an anti-CD20 antibody, wherein the patient had an NMOSD attack while being treated with the anti-CD20 antibody. The description provides for a method of treating NMOSD, the method comprising: administering an anti-CD19 antibody to a patient in need of treatment for NMOSD, wherein the patient had been previously treated with an anti-CD20 antibody, wherein the patient had an NMOSD attack within 6 months of the last dose of the anti-CD20 antibody.

The description provides for a method of treating NMOSD, the method comprising: administering the anti-CD19 antibody VIB551 to a patient in need of treatment for NMOSD, wherein the patient is an AQP4-IgG+ patient, wherein the VIB551 is administered intravenously with a first initial dose of 300 mg, a second initial dose of 300 mg two weeks after the first initial dose, and subsequent doses of 300 mg every 6 months following the first initial dose, wherein patient had been previously treated with an anti-CD20 antibody, and wherein the patient had an NMOSD attack while being treated with the anti-CD20 antibody. The description provides for a method of treating NMOSD, the method comprising: administering the anti-CD19 antibody VIB551 to a patient in need of treatment for NMOSD, wherein the patient is an AQP4-IgG+ patient, wherein the VIB551 is administered intravenously with a first initial dose of 300 mg, a second initial dose of 300 mg two weeks after the first initial dose, and subsequent doses of 300 mg every 6 months following the first initial dose, wherein the patient had been previously treated with an anti-CD20 antibody; and, wherein the patient had an NMOSD attack within 6 months of the last dose of the anti-CD20 antibody.

### BRIEF DESCRIPTION OF FIGURES

FIG. 1. Flow chart of the multiplicity adjustment strategy in N-MOmentum clinical trial.

FIG. 2. Study design flow diagram for N-MOmentum clinical trial. (AC, adjudication committee, FU, follow-up; i.v., intravenous; max, maximum; min, minimum; NMO/NMOSD, neuromyelitis optica/neuromyelitis optica spectrum disorder; OLP, open-label period; RCP, randomised controlled period; Q26, every 26 (weeks); SFP, safety follow-up period)

FIG. 3. N-MOmentum clinical trial CONSORT flow diagram. (*Efficacy endpoints were assessed in the intent-to-treat population, defined as participants who were randomised and received any investigational product and analysed according to their randomised treatment group, regardless of whether participants received an intervention other than the one planned. †Safety endpoints were assessed in the as-treated population, defined as participants who received any investigational product; however, participants randomised to VIB551 who received all placebo doses were included in the placebo group. Conversely, participants randomised to placebo who received at least one dose of VIB551 were included in the active treatment group. ‡Other includes one case each of need for treatment with prohibited medication, incorrect randomisation of an ineligible participant and withdrawal prior to dosing owing to occurrence of an attack on the day of randomisation (VIB551 arm), and patient decision (placebo arm). CONSORT, Consolidated Standards of Reporting Trials; i.v., intravenous, RCP, randomised controlled period.)

FIG. 4. VH (SEQ ID NO:1) and VL (SEQ ID NO:2) amino acid sequences of the VIB551 antibody.

FIG. 5. Attack history for N-MOmentum participants with prior Rituximab usage.

FIG. 6. Attack-free probability stratified by prior Rituxumab use.

FIG. 7. Absolute CD2N+ B-cell counts of individual participants with prior rituximab use during the randomized control period (weeks 0-28) and open-label extension (>week 28). All participants received inebilizumab during the open-label extension.

FIG. 8. IgG concentrations at baseline and with inebilizumab treatment in participants with and without prior use of rituximab. IgG, immunoglobulin G.

FIG. 9. Heavy (SEQ ID N0:3) and light (SEQ ID NO:4) chain amino acid sequences of the VIB551 antibody.

### DETAILED DESCRIPTION

Described herein are methods for treating patients diagnosed with NMOSD using VIB551 (also referred to MEDI551, Uplizna® or Inebilizumab), wherein the patients have received a previous anti-CD20 antibody treatment. Also described herein are methods for reducing active MRI lesions in a patient diagnosed with NMOSD using VIB551, wherein the patients have received a previous anti-CD20 antibody treatment. Additionally described herein are methods of reducing AQP4-IgG titers in a AQP4-IgG⁺ patient in need of treatment for NMOSD using VIB551, wherein the patients have received a previous anti-CD20 antibody treatment. Further described herein are methods of reducing NMOSD-related disability in a patient diagnosed with NMOSD using VIB551, wherein the patients have received a previous anti-CD20 antibody treatment. Also described herein are methods of reducing NMOSD-related attacks in a patient in need of treatment for NMOSD using VIB551, wherein the patients have received a previous anti-CD20 antibody treatment.

In aspects, in of the methods described herein. VIB551 may be used to treat patients diagnosed with NMOSD who had one or more NMOSD attacks while being treated with the anti-CD20 antibody. In aspects, VIB551 may be used to treat patients diagnosed with NMOSD that had one or more NMOSD attacks within 6 months of the last dose of the anti-CD20 antibody. In particular aspects, the patient may be an astrocyte water channel aquaporin-4 (AQP4)-IgG+ patient. In aspects, the anti-CD20 antibody, with which the patient may have been previously treated with, is rituximab (antibody C2B8 in WO94/11026, which is incorporated herein by reference in its entirety). In aspects, the anti-CD20 antibody, with which the patient may have been previously treated with, is ABP-300 (Abpro); B-001 (Shanghai Pharmaceuticals Holding); BAT-4306F (Bio-Thera Solutions); BAT-4406F (Bio-Thera Solutions); BCD-132 (Biocad Biotechnology); BVX-20 (Vaccinex); CYT-202 (Cytovia Therapeutics); epcoritamab (Genmab); GB-261 (Genor Biopharma); GD-CO1620 (ManysmarT); glofitamab (Roche); HS-006 (Zhejiang Hisun Pharmaceutical); IGM-2323 (IGM Biosciences); IMM-0306 (ImmuneOnco Biopharma); MIL-62 (Beijing Mabworks Biotech); mosunetuzumab (Roche); MRG-001 (Shanghai Miracogen); obinutuzumab (Roche); ocrelizumab (Roche; Niogen); odronextamab (Regeneron); ofatumumab (Genmab; Novartis); plamotamab (Xencor); SM-09 (SinoMab BioScience); TRS-005 (Zhejiang Teruisi Biopharma); ublituximab (rEVO Biologics); or YBL-031 (Y-Biologics).

In aspects, the disclosure provides a method of treating neuromyelitis optica spectrum disorder (NMOSD), the method comprising administering the anti-CD19 antibody VIB551 to a patient in need of treatment for NMOSD, wherein the patient had been previously treated with an anti-CD20 antibody, and wherein the patient had an NMOSD attack while being treated with the anti-CD20 antibody. In aspects, the disclosure provides a method of treating NMOSD, the method comprising: administering the anti-CD19 antibody VIB551 to a patient in need of treatment for NMOSD, wherein patient had been previously treated with an anti-CD20 antibody, wherein the patient had an NMOSD attack within 6 months of the last dose of the anti-CD20 antibody.

If VIB551 is used to treat NMOSD, it may treat the NMOSD by reducing the worsening of the patient's Kurtzke Expanded Disability Severity Scale (EDSS), or by reducing the number of active magnetic resonance imaging (MRI) lesions in the patient, or by reducing the worsening of the modified Rankin score of the patient, or by reducing the frequency of in-patient hospitalization of the patient related to NMOSD, or by reducing the risk of an NMOSD-related attack of the patient, or by reducing optic neuritis, or by reducing the severity of the patient's NMOSD-related attacks, or by decreasing the patient's pain, or by reducing NMOSD-related damage in the patient, or by reducing NMOSD-related attacks in the patient.

If the VIB551 treats the patient's NMOSD by reducing the worsening of the patient's EDSS score, and the patient has a baseline EDSS score of 0, then the patient's EDSS score may worsen by fewer than 2 points, or worsen by fewer than 1 point, or worsen by fewer than 0.5 points. This reduction of worsening of EDSS score for the patient with the baseline score of 0 may be over a time period of at least 6 months, 9 months, 1 year, 2 years, 3 years, 4 years, 5 years, 7.5 years, or 10 years. If the VIB551 treats the patient's NMOSD by reducing the worsening of the patient's EDSS score, and the patient has a baseline score of 1 to 5, then the patient's EDSS score may worsen by fewer than 1 point, or worsen by fewer than 0.5 point. This reduction of worsening for the patient with the baseline EDSS score of 1 to 5 may be reduction of worsening over a time period of over 6 months, 9 months, 1 year, 2 years, 3 years, 4 years, 5 years, 7.5 years, or 10 years. If the VIB551 treats the patient's NMOSD by reducing the worsening of the patient's EDSS score, and the patient has a baseline EDSS score of 5.5 or more, then the patient's EDSS score may worsen by fewer than 0.5 point or worsen by fewer than 0.25 point. This reduction of worsening for the patient with the baseline score of 5.5 or more may be reduction of worsening of EDSS score over a time period of over 6 months, 9 months, 1 year, 2 years, 3 years, 4 years, 5 years, 7.5 years, or 10 years.

If the VIB551 treats the patient's NMOSD by reducing the number of active MRI lesions, then the treating may be a reduction in the number of enlarging T2 MRI lesions or may be a reduction in the number of new MRI lesions, or may be a reduction in both the number of enlarging T2 MRI lesions and the number of new MRI lesions. The reduction in lesions may be a reduction in brain lesions, a reduction in brainstem lesions, a reduction in spinal cord lesions, a reduction in optic nerve lesions, or a reduction lesions in a combination of any two or more of brain, brainstem, spinal cord, and optic nerve. The new MRI lesions may not be clinically symptomatic.

If the VIB551 treats the patient's NMOSD by reducing the worsening of the patient's modified Rankin score, then the reducing of worsening may be such that the patient's modified Rankin score worsens by fewer than 2 points or by fewer than 1 point over a time period of at least 6 months, or of at least 9 months, or at least 1 year, or at least 2 years, or at least 3 years, or at least 4 years, or at least 5 years, or at least 7.5 years, or at least 10 years.

If the VIB551 treats the patient's NMOSD by reducing the risk of an NMOSD-related attack, then the patient's risk of attack may be reduced by between 60% and 85%, or may be reduced by between 65% and 75%, or may be reduced by between 70% and 80%. The patient's risk of attack may be reduced by at least 70%, at least 75%, at least 76%, at least 77%, at least 78%, at least 79% or at least 80%. The patient's risk of attack may be reduced by 70%, 75%, 76%, 77%, 78%, 79% or 80%.

If the VIB551 treats the patient's NMOSD by reducing the risk of an NMOSD-related attack, then as a result of the reduction in risk of an NMOSD-related attack, the treated patient's probability of having no NMOSD-related attack may be greater than 70% over at least 6 months following VIB551 treatment, or greater than 70% over at least 12 months following VIB551 treatment, or greater than 70% over at least 18 months following VIB551 treatment. As a result of the reduction in risk of an NMOSD-related attack, the treated patient's probability of having no NMOSD-related attack may be greater than 75% over at least 6 months following VIB551 treatment, or greater than 75% over at least 12 months following VIB551 treatment, or greater than 75% over at least 18 months following VIB551 treatment. In addition, as a result of the reduction in risk of an NMOSD-related attack, the treated patient's probability of having no NMOSD-related attack may be greater than 80% over at least 6 months following VIB551 treatment, or greater than 80% over at least 12 months following VIB551 treatment, or greater than 80% over at least 18 months following VIB551 treatment. Also, as a result of the reduction in risk of an NMOSD-related attack, the treated patient's probability of having no NMOSD-related attack may be greater than 85% over at least 6 months following VIB551 treatment, or greater than 85% over at least 12 months following VIB551 treatment, or greater than 85% over at least 18 months following VIB551 treatment.

Furthermore, if the VIB551 treats the patient's NMOSD by reducing the risk of an NMOSD-related attack, then as a result of the reduction in risk, the treated patient's annualized risk of an NMOSD-related attack may be reduced to between 0.18 and 0.08, or it may be reduced to between 0.15 and 0.08, or it may be reduced to 0.14, or 0.13, or 0.12, or 0.11, or 0.10, or 0.09, or 0.08, or 0.07. If the patient in treatment for NMOSD is AQP4-IgG seropositive, then the patient's annualized risk of an NMOSD-related attack may be reduced to between 0.15 and 0.11, or reduced to between 0.14 and 0.12, or it may be reduced to 0.14, 0.13, 0.12, or 0.11. If the patient in treatment for NMOSD is AQP4-IgG seronegative, then the patient's annualized risk of an NMOSD-related attack may be reduced to between 0.09 and 0.07, or it may be reduced to 0.09, 0.08, or 0.07.

The NMOSD-related attack, the risk of which may be reduced as in the treating of the NMOSD patient, may be an attack characterized by the appearance of a new NMOSD symptom or the worsening of an existing NMOSD symptom. The new or existing symptom may be an eye symptom. If the new or existing symptom is an eye symptom it may be eye pain, a new optic nerve lesion, an enlarging optic nerve lesion, blurred vision, loss of vision, or a 5 or more character drop in low-contrast Landolt C Broken Rings Chart. The new or existing symptom may be a spinal cord symptom. If the new or existing symptom is a spinal cord symptom, it may be a deep or radicular pain, extremity paraesthesia, weakness, sphincter dysfunction, Lhermitte's sign, a new spinal cord lesion, or an enlarging spinal cord lesion. The new or existing symptom may be a brain or brain stem symptom. If the new or existing symptom is a brain or brain stem symptom, it may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. The new or worsening symptom may a combination of any two or more of the eye, spinal cord, or brain/brain stem symptoms. It may be a combination of any two, three, or four of these symptoms.

If the VIB551 treats the patient's NMOSD by reducing optic neuritis, then the patient may experience reduced eye pain, reduced vision loss, reduced visual field loss, reduced loss of color vision, or reduced flashing or flickering of lights with movement of the eye. The reduction in optic neuritis may result in improved vision, and/or relief from eye pain.

If the VIB551 treats the patient's NMOSD by reducing the severity of the patient's NMOSD-related attacks, then the severity of any NMOSD-related attack suffered by the patient may be graded mild or moderate as opposed to graded as severe. A mild attack may be an attack that is transient, may be an attack that requires only minimal treatment or therapeutic intervention, and/or is an attack that may not interfere with usual activities of daily living. A moderate attack may be an attack that may be alleviated with specific additional therapeutic interventions. Any moderate attack may be one that interferes with usual activities of daily living, and/or causes discomfort, but that poses no significant or permanent risk of harm to the patient. The reduction of severity of the patient's NMOSD-related attacks may be a reduction in the attacks suffered by the patient as being graded as major. Such a major attack may be an attack that requires intensive therapeutic intervention, interrupts usual activities of daily living, or that significantly affects the clinical status of the patient. Such a major attack may require in-patient hospitalization.

If the VIB551 treats the patient's NMOSD by decreasing the patient's pain, then the decrease may be determined by a decrease in pain in the patient's eyes, legs, arms, upper back, and/or lower back. The decrease in pain may be in any one, any two, any three, any four, or all five of these regions. The decrease in pain may be measured by the pain numeric rating scale (PRS). The decrease in pain may be monitored relative to a baseline PRS level over a scale of 1 to 10. The decrease in pain may be a decrease in pain of at least one on the scale, at least 2 on the scale, at least 3 on the scale, at least 4 on the scale, or at least 5 on the scale. The decrease in pain may be a decrease in pain of between 1-5 on the scale, or a decrease in pain of between 1-3 on the scale, or a decrease in pain of between 1-2 on the scale.

If VIB551 treats the patient's NMOSD by reducing NMOSD-related damage, then the NMOSD-related damage may be the occurrence of a clinically asymptomatic new MRI lesion in the patient. If the NMOSD-related damage is the occurrence of a clinically asymptomatic new MRI lesion, it may occur in a patient who has not experienced symptoms of an NMOSD-related attack or in a patient who has experienced symptoms of an NMOSD-related attack.

If the clinically asymptomatic new MRI lesion occurs in a patient who has not experienced symptoms of an NMOSD-related attack, VIB551 may reduce the occurrence, or likelihood of occurrence, of a new clinically asymptomatic MRI lesion in any one or more domains, e.g., brain/brain stem, optic nerve or spinal cord, of the patient. The reduced occurrence, or likelihood of occurrence, of the new clinically asymptomatic MRI lesion may be a prevention of occurrence of the new clinically asymptomatic MRI lesion. The patient who has not experienced symptoms of an NMOSD-related attack, and whose NMOSD-related damage is reduced by VIB551, may be one who has not experienced symptoms of an NMOSD-related attack for at least 3 months, at least 6 months, at least 9 months, at least 12 months, at least 15 months, at least 18 months, at least 21 months or at least 24 months. Administration of VIB551 to the patient who has not experienced symptoms of an NMOSD-related attack may reduce the occurrence, or likelihood of occurrence, of a new clinically asymptomatic MRI lesion for the duration of the patient's treatment with VIB551. The administration of VIB551 to the patient who has not experienced symptoms of an NMOSD-related attack may result in a reduced occurrence, or likelihood of occurrence, of a new MRI lesion beginning within 1 month, within 2 months, or within 3 months following administration of a first dose of VIB551 and may continue for at least 6 months, at least 12 months, at least 18 months, at least 24 months, at least 30 months, at least 36 months, at least 42 months, at least at least 48 months, at least 54 months or at least 60 months following administration of the first dose of VIB551.

If the clinically asymptomatic new MRI lesion occurs in a patient who has experienced symptoms of an NMOSD-related attack, then the VIB551 may reduce the occurrence, or likelihood of occurrence, of a new clinically asymptomatic MRI lesion in a domain other than the domain in which the patient has experienced symptoms of the NMOSD-related attack. For instance, if the patient experienced symptoms of an NMOSD-related attack in the spinal cord, VIB551 may reduce the occurrence, or likelihood of occurrence, of a new MRI lesion in the optic nerve or brain/brain stem or both. If the VIB551 reduces, or reduces likelihood of, occurrence of a new clinically asymptomatic MRI lesion in association with an NMOSD-related attack, it may completely reduce, i.e., prevent, the occurrence of the new clinically asymptomatic MRI lesion in the patient. Moreover, VIB551 may not only reduce, or reduce likelihood of, a new clinically asymptomatic MRI lesion in the patent experiencing symptoms of the NMOSD-related attack, it may also reduce, or reduce likelihood of, occurrence of a new MRI lesion in the domain in which the patient experiences the symptoms of the NMOSD-related attack.

VIB551 may also be used in a method of reducing active MRI lesions in a patient diagnosed with NMOSD. In aspects, the disclosure provides a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. In aspects the disclosure provides a method of reducing active MRI lesions in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. If the VIB551 is used in a method of reducing active MRI lesions in a patient, then the VIB551 may reduce the cumulative total of new and enlarging lesions in the patient. If the VIB551 is used in a method of reducing active MRI lesions in a patient diagnosed with NMOSD, then the VIB551 may reduce the cumulative total of new gadolinium [Gd]-enhancing lesions, new T2 lesions and enlarging T2 lesions in the patient. If the VIB551 is used in a method of reducing active MRI lesions in a patient diagnosed with NMOSD, then the VIB551 may reduce the number of new T2 lesions in the patient and the number of enlarging T2 lesions in the patient. If the VIB551 is used in a method of reducing active MRI lesions in a patient diagnosed with NMOSD, then the VIB551 may reduce the number of new T2 lesions in the patient or the number an enlarging T2 lesions in the patient. The active MRI lesions reduced in the patient may be lesions in the brain/brain stem, spinal cord, and optic nerve cumulatively, or may be lesions in one or two of the brain/brain stem, spinal cord, or optic nerve. The active MRI lesions reduced in the patient may be clinically symptomatic or clinically asymptomatic MRI lesions. If the MRI lesions are clinically asymptomatic, they may be new MRI lesions that occur in a patient who has not experienced symptoms of an NMOSD-related attack. If the MRI lesions are clinically asymptomatic, they may be new MRI lesions that occur in a patient in association with an NMOSD-related attack, but not in the same domain as the domain in which the patient experiences the symptoms of the NMOSD-related attack.

The VIB551 may also be used in a method of reducing AQP4-IgG titers in a AQP4-IgG⁺ patient in need of treatment for NMOSD. In aspects, the disclosure provides a method of reducing AQP4-IgG titers in a AQP4-IgG+ patient in need of treatment for NMOSD, the method comprising administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody; and wherein the VIB551 is administered intravenously at a dose of 300 mg every 6 months. In aspects, the disclosure provides a method of reducing AQP4-IgG titers in a AQP4-IgG+ patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody; and, wherein the VIB551 is administered intravenously at a dose of 300 mg every 6 months. If the VIB551 is used in a method of reducing AQP4-IgG titers in a AQP4-IgG⁺ patient in need of treatment for NMOSD, then the VIB551 may reduce the AQP4-IgG titers by 75% to 100%, or by 75% to 90%, or by 75% to 85%, or by 80% to 100%, or by 85% to 100%, or by 90% to 95%, or by 75%, 80%, 85%, 90%, 95 or 100%. The VIB551 may reduce the AQP4-IgG titers for a sustained period of time of at least 2 months, at least 3 months, at least 4 months, at least 5 months, at least 6 months, at least 7 months, at least 8 months, at least 9 months, at least 10 months, at least 11 months or at least 12 months following administration of a VIB551 dose.

The VIB551 may also be used in a method of reducing NMOSD-related disability in a patient diagnosed with NMOSD. In aspects, the disclosure provides a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. In aspects, the disclosure provides a method of reducing NMOSD-related disability in a patient diagnosed with NMOSD, the method comprising: administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. The reducing of the NMOSD-related disability in the patient may be a reduction in the worsening of NMOSD-related disability in the patient or it may be a lessening of NMOSD-related disability in the patient. The NMOSD-related disability may be neurological disability or a manifestation of a neurological disability. The NMOSD-related disability may be characterized by one or more of eye pain, a loss of color vision, an overall loss of vision, blurred vision, double vision, overall weakness or paralysis, weakness or paralysis in the arms or legs, radicular pain, uncontrollable hiccups, uncontrollable nausea or vomiting, loss of bladder or bowel control, paralysis, and/or fatigue.

The reducing the NMOSD-related disability may be determined using EDSS, or may be determined using the modified Rankin Scale (mRS), or may be determined using EDSS and mRS. The reducing the NMOSD-related disability may be detectable within 6 to 12 months, within 6 to 8 months, or within 6 to 7 months of administering a dose of VIB551 or administering a first dose of VIB551.

If the reducing the NMOSD-related disability is determined using EDSS, the reducing in NMOSD-related disability may be either a reduction in the worsening in the patient's EDSS score or a lowering of the patient's EDSS score.

If the reducing the NMOSD-related disability is a reduction in the worsening in the patient's EDSS score, and the patient has a baseline EDSS score of 0, then the reduction in worsening may be such that, if the patient's EDSS score worsens, it worsens to a score of 0.5, or to a score of no more than 1, or to a score of no more than 1.5, or to a score of nor more than 2 over a period of time. The period of time in which the patient with the baseline score of 0 worsens to a score of 0.5, to no more than 1, to no more than 1.5, or to no more than 2 may be a period of time of at least 6 months, 9 months, 1 year, 1.5 years, 2 years, 3 years, 4 years, 5 years, 7.5 years, or 10 years. If the reducing the NMOSD-related disability is a reduction in the worsening in the patient's EDSS score, and the patient has a baseline EDSS score of 1 to 5, then the reduction in worsening may be a worsening of the patient's EDSS score by 0.5 points or by no more than 1 point over a period of time. The period of time in which the patient with the baseline score of 1 to 5 worsens by 0.5 points, or by no more than 1 point, may be a period of time of at least 6 months, 9 months, 1 year, 1.5 years, 2 years, 3 years, 4 years, 5 years, 7.5 years, or 10 years. If the reducing the NMOSD-related disability is a reduction in the worsening in the patient's EDSS score, and the patient has a baseline EDSS score of 5.5 or more, then the reduction in worsening may be a worsening of the patient's EDSS score by no more than 0.5 points. The period of time in which the patient with the baseline score of 5.5 worsens by the no more than 0.5 points may be a period of time of at least 6 months, 9 months, 1 year, 1.5 years, 2 years, 3 years, 4 years, 5 years, 7.5 years, or 10 years. The patient's baseline EDSS score may be determined within approximately 1 month, 2 weeks, 1 week, 3 days, 2 days, or 1 day of administration of a first VIB551 dose. The patient's baseline EDSS score may be determined coincident with administration of a first VIB551 dose or it may be determined within 1 day, within 2 days, within 3 days, within 1 week, within 2 weeks, or within 1 month of administration of a first VIB551 dose.

If the reducing the NMOSD-related disability is a lowering of the patient's EDSS score, then the patient's EDSS score may decrease by at least 0.5 points, or at least 1 point, or at least 1.5 points, or at least 2 points. The lowering or decreasing of the patient's EDSS score by the at least 0.5, at least 1, at least 1.5, or at least 2 points may occur over a period of time of approximately 2 weeks, approximately 1 month, approximately 2 months, approximately 3 months, approximately 4 months, approximately 5 months, approximately 6 months, approximately 9 months, approximately 12 months, or approximately 18 months. The period of time over which the patient's EDSS score is lowered, or that the patient's EDSS score decreases, may begin within 1 month, 2 weeks, 1 week, 3 days, 2 days, or 1 day of administration of a first VIB551 dose; or it may be coincident with administration of a first VIB551 dose; or it may begin within 1 day, within 2 days, within 3 days, within 1 week, within 2 weeks, or within 1 month of administration of a first VIB551 dose. The period of time over which the patient's EDSS score is lowered or decreases may begin at the time of an NMOSD attack, or may begin within 1 day, 2 days, 3 days, 4 days, 5 days, 6 days, or 7 days from the time of an NMOSD attack.

If the reducing the NMOSD-related disability is determined using mRS, then the reducing in NMOSD-related disability may be either a reduction in the worsening in the patient's mRS score or a lowering of the patient's mRS score.

If the reducing the NMOSD-related disability is a reduction in the worsening in the patient's mRS score, the reduction in worsening in mRS score may be a worsening of the patient's baseline mRS score by no more than 0.5 points or by no more than 1 point, or by no more than 1.5 points, or by no more than 2 points over a period of time. The period of time in which the patient's baseline mRS score worsens by the no more than 0.5 points, no more than 1 point, no more than 1.5 point, or no more than 2 points may be a period of time of at least 6 months, 9 months, 1 year, 1.5 years, 2 years, 3 years, 4 years, 5 years, 7.5 years, or 10 years. The patient's baseline mRS score, from which the reduction in worsening is determined, may be the patient's mRS score at approximately 1 month, approximately 2 weeks, approximately 1 week, approximately 3 days, approximately 2 days, or approximately 1 day prior to administration of a first VIB551 dose. The patient's baseline mRS score may be the patient's mRS score at the time of administration of a first VIB551 dose or it may be the patient's mRS score within 1 day, within 2 days, within 3 days, within 1 week, within 2 weeks, or within 1 month of administration of a first VIB551 dose.

If the reducing the NMOSD-related disability is a lowering of the patient's mRS score, then the patient's mRS score may lower or decrease by at least 0.5 points, or at least 1 point, or at least 1.5, or at least 2 points. The lowering or decrease of the patient's mRS score by the at least 0.5, at least 1, at least 1.5, or at least 2 points may be a lowering or decrease that occurs over a period of time of approximately 2 weeks, approximately 1 month, approximately 2 months, approximately 3 months, approximately 4 months, approximately 5 months, approximately 6 months, approximately 9 months, approximately 12 months, or approximately 18 months. The period of time over which the patient's mRS score is lowered may begin within 1 month, 2 weeks, 1 week, 3 days, 2 days, or 1 day of administration of a first VIB551 dose; or it may be coincident with administration of a first VIB551 dose; or it may occur within 1 day, within 2 days, within 3 days, within 1 week, within 2 weeks, or within 1 month of administration of a first VIB551 dose. The period of time over which the patient's mRS score is lowered may begin at the time of an NMOSD attack, or may begin within 1 day, 2 days, 3 days, 4 days, 5 days, 6 days, or 7 days from the time of an NMOSD attack.

The VIB551 may also be used in a method of reducing NMOSD-related attacks in a patient in need of treatment for NMOSD, wherein the patient has previously received an anti-CD20 antibody. In aspects, the disclosure provides a method of reducing NMOSD-related attacks in a patient in need of treatment for NMOSD, the method comprising: administering the anti-CD19 antibody VIB551 to a patient in need of treatment for NMOSD, wherein the patient had been previously treated with an anti-CD20 antibody, wherein the patient had an NMOSD attack while being treated with the anti-CD20 antibody. In aspects, the disclosure provides a method of reducing NMOSD-related attacks in a patient in need of treatment for NMOSD, the method comprising: administering the anti-CD19 antibody VIB551 at a dose of about 300 mg to a patient in need of treatment for NMOSD, wherein the patient had been previously treated with an anti-CD20 antibody, wherein the patient did not have an NMOSD attack while being treated with the anti-CD20 antibody. For example, the prior to VIB551 administration, the patient may have received anti-CD20 antibody therapy for up to 1 month, up to 2 months, up to 4 month, up to 6 months, up to 12 months, up to 2 years, up to 3 years, up to 4 years, up to 5 years, up to 6 years, or longer. Typically, the prior anti-CD-20 antibody is received for at least about 12 months prior to administering VIB551. The VIB551 may be administered in a dose of about 300 mg VIB551, optionally followed by one or more subsequent additional doses of 300 mg VIB551 every 6 months thereafter.

The VIB551 may also be used in methods of treating NMOSD in a patient. In aspects, the method comprises administering the anti-CD19 antibody VIB551 to a patient in need of treatment for NMOSD, optionally as a monotherapy, wherein the patient had been previously treated with an anti-CD20 antibody for at least 12 months and up to 5 years, and wherein one or more doses of about 300 mg of the anti-CD19 antibody VIB551 is administered after cessation of anti-CD20 therapy, each dose of VIB551 being administered about 6 months apart.

In aspects, the method of treating NMOSD in a patient comprises administering the anti-CD19 antibody VIB551 to a patient in need of treatment for NMOSD, optionally as a monotherapy, wherein the patient had been previously treated with an anti-CD20 antibody, wherein following 1, 2 or 3 NMOSD-related attacks the anti-CD20 therapy was stopped and wherein one or more doses of about 300 mg of the anti-CD19 antibody VIB551 is administered after the cessation of anti-CD20 therapy, each dose of VIB551 being administered about 6 months apart.

In aspects, the disclosure provides a method of reducing NMOSD-related attacks in a patient in need of treatment for NMOSD, the method comprising, administering the anti-CD19 antibody VIB551 to a patient in need of treatment for NMOSD, wherein the patient had been previously treated with an anti-CD20 antibody, wherein the patient had an NMOSD attack within 6 months of the last dose of the anti-CD20 antibody. The reducing of the NMOSD-related attacks in the patient may be a reduction in number of NMOSD-related attacks suffered by the patient in a first time period relative to a second time period. The first time period may occur following administration of a first VIB551 dose and the second time period may occur preceding the administration of the first VIB551 dose. The first time period may begin immediately following administration of a first VIB551 dose and the second time period may end immediately preceding the administration of the first VIB551 dose. The first and the second time period may be of an equal length in time. For example, the first and the second time period may both be at least 6 months, or 6 months, or at least 12 months, or 12 months, or at least 18 months, or 18 months, or at least 24 months, or 24 months, or at least 30 months, or 30 months, or at least 36 months, or 36 months, or at least 42 months, or 42 months, or at least 48 months, or 48 months, or at least 54 months or 54 months, or at least 60 months or 60 months.

The reduction in number of NMOSD-related attacks suffered by the patient in the first time period relative to the second time period may such that the patient suffers at least 1 fewer attack, or 1 fewer attack, or at least 2 fewer attacks, or 2 fewer attacks, or at least 3 fewer attacks, or 3 fewer attacks, or at least 4 fewer attacks, or 4 fewer attacks, or at least 5 fewer attacks, or 5 fewer attacks in the first time period relative to the second time period. The NMOSD-related attacks suffered by the patient in the first and/or the second time period may be NMOSD-related attacks that are optic neuritis attacks, myelitis attacks, or brainstem attacks. If the patient suffers an NMOSD-related attack in the first time period, the NMOSD-related attack may or may not be in the same domain, e.g., optic nerve, spinal cord or brain/brain stem, as an NMOSD-related attack(s) suffered by the patient in the second time period.

The NMOSD-related attacks suffered by the patient, the number of which may be reduced in the patient in need of treatment for NMOSD, may be NMOSD-related attacks characterized by the appearance of a new NMOSD symptom, or the worsening of an existing NMOSD symptom, or the appearance of a new MRI lesion that may or may not be symptomatic.

If the NMOSD-related attack is an attack characterized by a new symptom or the worsening of an existing symptom, the new or worsening symptom may be an eye symptom. If the new or worsening symptom is an eye symptom it may be eye pain, a new optic nerve lesion, an enlarging optic nerve lesion, blurred vision, loss of vision, or a 5 or more character drop in low-contrast Landolt C Broken Rings Chart. The NMOSD-related attack, characterized by a new eye symptom or the worsening of an existing eye symptom, may further/alternatively meet any one or more of the following criteria: >15-character drop in high-contrast Landolt C Broken Ring Chart from most recent clinical visit as measured in a previously affected eye and no other ophthalmological explanation, Reduction of ≥2 steps in CF (counting fingers) to NLP (no light perception) from most recent clinical visit as measured in a previously affected eye and no other ophthalmological explanation, Reduction of 7 characters in low-contrast Landolt C Broken Ring Chart from most recent clinical visit as measured in either eye alone (monocular) and a new RAPD (relative afferent pupillary defect) in affected eye. Reduction of ≥7 characters in low-contrast Landolt C Broken Ring Chart from most recent clinical visit as measured in either eye alone (monocular) and loss of a previously documented RAPD in fellow eye, Reduction of ≥5 characters in high-contrast Landolt C Broken Ring Chart from most recent clinical visit as measured in either eye alone (monocular) and a new RAPD in affected eye, Reduction of 25 characters in high-contrast Landolt C Broken Ring Chart from most recent clinical visit as measured in either eye alone (monocular) and loss of a previously documented RAPD in fellow eye, Reduction of ≥1 step§ in CF to NLP from most recent clinical visit as measured in a previously affected eye and a new RAPD in affected eye, Reduction of ≥1 step§ in CF to NLP from most recent clinical visit as measured in a previously affected eye and loss of a previously documented RAPD in fellow eye, Reduction of 7 characters in low-contrast Landolt C Broken Ring Chart from most recent clinical visit as measured in either eye alone (monocular) and a new Gd-enhancing or new/enlarging T2 MRI lesion in the corresponding optic nerve. Reduction of ≥5 characters in high-contrast Landolt C Broken Ring Chart from most recent clinical visit as measured in either eye alone (monocular) and a new Gd-enhancing or new/enlarging T2 MRI lesion in the corresponding optic nerve, Reduction of 21 step§ in CF to NLP from most recent clinical visit as measured in a previously affected eye and a new Gd-enhancing or new/enlarging T2 MRI lesion in the corresponding optic nerve.

If NMOSD-related attack is an attack characterized by a new symptom or the worsening of an existing symptom, the new or worsening symptom may be a spinal cord symptom. If the new or worsening symptom is a spinal cord symptom, it may be a deep or radicular pain, extremity paraesthesia, weakness, sphincter dysfunction, Lhermitte's sign, a new spinal cord lesion, or an enlarging spinal cord lesion. The NMOSD-related attack, characterized by a new spinal cord symptom or the worsening of an existing spinal cord symptom, may further/alternatively meet any one or more of the following criteria: Worsening of ≥2 points in at least one of the relevant (pyramidal, bladder/bowel, sensory) FSS compared with most recent clinical visit, Worsening of ≥1 point in EDSS score compared with most recent clinical visit if previous EDSS score ≥5.5, Worsening of ≥1 point in at least two of the relevant (pyramidal, bladder/bowel, sensory) FSS compared with most recent clinical visit when the most recent clinical visit score was ≥1 and a new Gd-enhancing or new/enlarging T2 MRI lesion in the spinal cord, Worsening of ≥0.5 points in EDSS score compared with most recent visit if previous EDSS score ≥5.5 and a new GD-enhancing or new/enlarging T2 MRI lesion in the spinal cord.

If NMOSD-related attack is an attack characterized by a new symptom or the worsening of an existing symptom, the new or worsening symptom may be a brain or brain stem symptom. If the new or existing symptom is a brain or brain stem symptom, it may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. The NMOSD-related attack, characterized by a new brain/brain stem symptom or the worsening of an existing brain/brain stem symptom, may further/alternatively meet any one or more of the following criteria: Isolated (not present at most recent clinical visit) intractable nausea, vomiting, and/or hiccups lasting ≥48 hours and a new Gd-enhancing or new/enlarging T2 MRI lesion in the brainstem. Worsening of ≥2 points in at least one of the relevant (brainstem, cerebellar) FSS compared with most recent clinical visit and a new Gd-enhancing or new/enlarging T2 MRI lesion in the brainstem, or Worsening of ≥2 points in at least one of the relevant (cerebral, sensory, pyramidal) FSS (with a score of ≥3 at the current visit) compared with most recent clinical visit and a new Gd-enhancing or new/enlarging T2 MRI lesion in the brain consistent with the clinical presentation.

The NMOSD-related attack may be an attack characterized by any combination of new and/or worsening symptoms of any one, two, or more of the eye, spinal cord, or brain/brain stem. The NMOSD-related attack may be an attack characterized by any combination of two, three, or four of the symptoms or other criteria identified for any of the one or more of the eye, spinal cord or brain/brain stem.

Furthermore, the NMOSD-related attack may be an attack characterized by the appearance of new MRI lesions in the patient. The new MRI lesions may be, but are not necessarily, symptomatic.

The NMOSD patient to which the VIB551 is administered in the methods disclosed herein may or may not be AQP4-IgG seropositive. The NMOSD patient may be screened for AQP4-IgG prior to VIB551 administration. In aspects, VIB551 may be administered to a patient with an increase in serum Nfl levels over a baseline level in any of the methods disclosed herein (as described in U.S. Prov. Appl. Nos. 63/052,093 and 63/071,092, which are incorporated herein by reference in their entirety) In aspects, VIB551 is administered to a patient with a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater. (as described in U.S. Prov. Appl. No. 63/046,133, which is incorporated herein by reference in its entirety). If VIB551 is administered to the patient with the sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater, the patient's sGFAP concentration may be determined to be about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater prior to the administering.

In aspects, an anti-CD19 antibody, other than V1B551, e.g., MOR00208 (also referred to as Xmab 5574 or tafasitamab; disclosed in U.S. Patent Application No. 20170137516), may be used in the methods of treatment disclosed herein. In certain of these aspects, the disclosure provides a method of treating NMOSD, the method comprising: administering an anti-CD19 antibody to a patient in need of treatment for NMOSD, wherein the patient had been previously treated with an anti-CD20 antibody, wherein the patient had an NMOSD attack while being treated with the anti-CD20 antibody. In aspects, the disclosure provides a method of treating NMOSD, the method comprising: administering an anti-CD19 antibody to a patient in need of treatment for NMOSD, wherein the patient had been previously treated with an anti-CD20 antibody, wherein the patient had an NMOSD attack within 6 months of the last dose of the anti-CD20 antibody. In aspects, the anti-CD20 antibody may be rituximab (antibody C2B8 in WO94/11026). In aspects, the anti-CD20 antibody the patient may previously have been treated with is ABP-300 (Abpro); B-001 (Shanghai Pharmaceuticals Holding); BAT-4306F (Bio-Thera Solutions); BAT-4406F (Bio-Thera Solutions); BCD-132 (Biocad Biotechnology); BVX-20 (Vaccinex); CYT-202 (Cytovia Therapeutics); epcoritamab (Genmab); GB-261 (Genor Biopharma); GD-CO1620 (ManysmarT); glofitamab (Roche); HS-006 (Zhejiang Hisun Pharmaceutical); IGM-2323 (IGM Biosciences); IMM-0306 (ImmuneOnco Biopharma); MIL-62 (Beijing Mabworks Biotech); mosunetuzumab (Roche); MRG-001 (Shanghai Miracogen); obinutuzumab (Roche); ocrelizumab (Roche; Niogen); odronextamab (Regeneron); ofatumumab (Genmab; Novartis); plamotamab (Xencor); SM-09 (SinoMab BioScience); TRS-005 (Zhejiang Teruisi Biopharma); ublituximab (rEVO Biologics); or YBL-031 (Y-Biologics).

In aspects, the anti-CD19 antibody administered to the patient, if other than VIB551 (also referred to MED1551, Uplizna™ or Inebilizumab; disclosed in U.S. application Ser. No. 11/852,106 and Int'l Appl. No. PCT/US20/29613, which are incorporated herein by reference in their entireties), may be any of, for example, MOR00208 (also referred to as Xmab 5574 or tafasitamab; disclosed in U.S. Patent Application No. 20170137516, which is incorporated herein by reference in its entirety); blinatumomab (Amgen; Astellas; MicroMet); loncastuximab tesirine (ADC Therapeutics); GTB-1550/OXS-1550 (Oxis Biotech Inc); obexelimab/XmAb5871 (Xencor Inc); AFMII (Affimed); or coltuximab/ravtansine (ImmunoGen Inc).

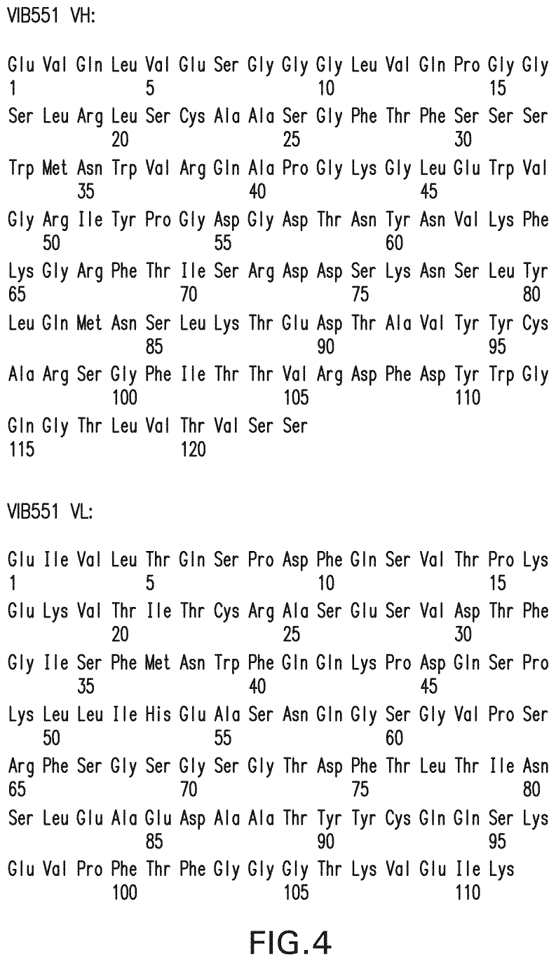

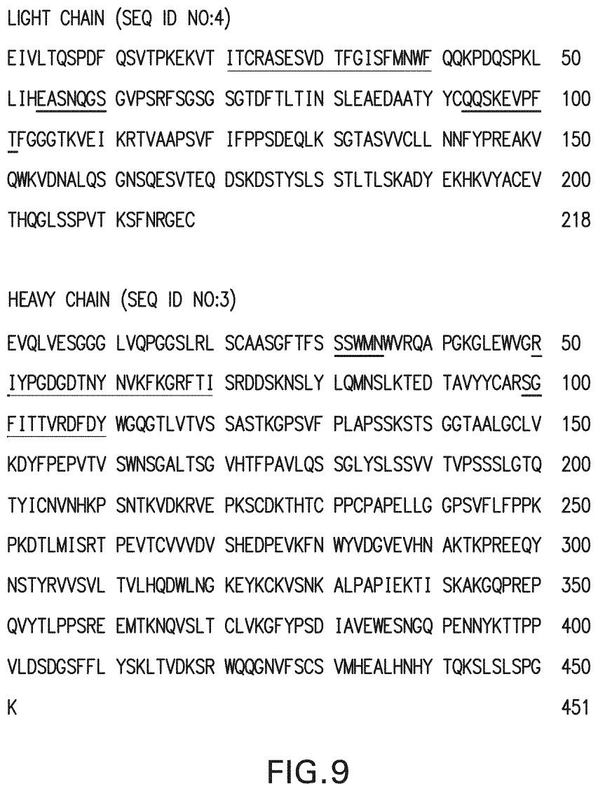

In aspects, if the anti-CD19 antibody VIB551 is administered in any of the methods disclosed herein, the VIB551 may have the VH amino acid sequence and a VL amino acid sequence as shown in FIG. 4 In aspects, the VIB551 may comprise a heavy chain variable region (VH) comprising the amino acid of SEQ ID NO:1 and alight chain variable region (VL) comprising the amino acid of SEQ ID NO: 2. The VIB551 administered in the methods may have the VH amino acid sequence and the VL amino acid sequence as shown in FIG. 4, but for one or more changes in amino acid residues that do not alter the function of the VIB551 amino acid sequence. The number of amino acid changes may be 1 amino acid residue change, 2 amino acid residue changes, 3 amino acid residue changes, 4 amino acid residue changes, or 5 amino acid residue changes. In particular aspects, the VIB551 used in the methods disclosed herein is at least about 90%, about 91%, about 92%, about 93%, about 94%, about 95%, about %%, about 97%, about 98%, about 99% or identical to the VH and VL sequences disclosed in FIG. 4. The VIB551 administered in the methods may contain the Complementarity Determining Region (CDR) amino acid sequences of the VH and the VL sequences as shown in FIG. 4 and Sequence Table 1, but may have one or more alterations in the framework regions (i.e residues other than the CDR residues) of the VH and the VL domain sequences shown in FIG. 4 and Table 1. Alternatively, the VIB551 administered in any of the methods disclosed herein may have a heavy chain amino acid sequence and a light chain amino acid sequence as shown in FIG. 9 In aspects, the VIB551 may comprise a heavy chain comprising the amino acid of SEQ ID NO:3 and a light chain comprising the amino acid of SEQ ID NO: 4. The VIB551 administered in the methods may have the heavy chain amino acid sequence and the light chain amino acid sequence as shown in FIG. 9, but for one or more changes in amino acid residues that do not alter the function of the VIB551 amino acid sequence. The number of amino acid changes may be 1 amino acid residue change, 2 amino acid residue changes, 3 amino acid residue changes, 4 amino acid residue changes, or 5 amino acid residue changes. In aspects, the VIB551 used in the methods disclosed herein is at least about 90%, about 91%, about 92%, about 93%, about 94%, about 95%, about 96%, about 97%, about 98%, about 99% or identical to the heavy chain and light chain sequences disclosed in FIG. 9.

TABLE 1
VIB551

| VH | | | | VL | | | |
|---|---|---|---|---|---|---|---|
| Domain | VH CDR1 | VH CDR2 | VH CDR3 | Domain | VL CDR1 | VL CDR2 | VL CDR3 |
| SEQ ID | SSWMN | RIYPGDGDTNYVN | SGFITTVRDFDY | SEQ ID | RASESVDTFGISFMN | EASNQGS | QQSKEVPFT |
| NO: 1 | (SEQ ID | KFKG | (SEQ ID NO: 7) | NO: 2 | (SEQ ID NO: 8) | (SEQ ID | (SEQ ID NO: 10) |
| | NO: 5) | (SEQ ID NO: 6) | | | | NO: 9) | |

In any method disclosed herein, if VIB551 is the anti-CD19 antibody administered to a patient in need, then the VIB551 may be administered at a dose of about 30) mg. In aspects, the VIB551 may be administered at a dose of about 250 mg to about 350 mg, about 275 mg to about 325 mg, about 290 mg to about 310 mg, about 205 mg to about 305 mg, or it may be a dose of 300 mg. In aspects, the patient may receive one or more initial doses of VIB551. In one aspect, the patient may receive, one, two, three or more initial doses. In particular aspects, the initial dose may be about 300 mg. In aspects, the VIB551 may be administered at an initial dose of about 250 mg to about 350 mg, about 275 mg to about 325 mg, about 290 mg to about 310 mg, about 205 mg to about 305 mg, or an initial dose of 300 mg. In particular aspects, VIB551 may be administered intravenously with a first initial dose of about 300 mg, a second initial dose of about 300 mg two weeks after the first initial dose, and subsequent doses of about 300 mg every 6 months following the first initial dose.

If VIB551 is administered in the methods disclosed herein, it may be administered at an interval of once about every 6 months. In aspects, the VIB551 may be administered intravenously. The approximately every 6 months may be administration every 6 months, every 180 days, every between 170 and 190 days, every between 175 and 185 days, every between 175 and 190 days, or every between 170 and 185 days. The approximately every 6 months may be administration every 26 weeks, every 25 weeks, every 27 weeks, every between 25 and 27 weeks, every between 25 and 26 weeks, or every between 26 and 27 weeks. Prior to the administering the VIB551 every approximately 6 months in the method, an initial VIB551 dose may be administered to the NMOSD patient. The initial VIB551 dose may be administered approximately 2 weeks before the approximately every 6 month VIB551 dosing. The administering the initial VIB551 dose approximately 2 weeks before the every approximately 6 month VIB551 dosing may be the administering of the initial VIB551 dose 12 days, 13 days, 14 days, 15 days, or 16 days before the approximately 6 months VIB551 dosing. The initial VIB551 dose may or may not be co-administered with oral corticosteroids.

In aspects, the disclosure provides a method of treating NMOSD, the method comprising: administering the anti-CD19 antibody VIB551 to a patient in need of treatment for NMOSD, wherein the patient is an AQP4-IgG+ patient, wherein the VIB551 is administered intravenously with a first initial dose of 300 mg, a second initial dose of 300 mg two weeks after the first initial dose, and subsequent doses of 300 mg every 6 months following the first initial dose, wherein patient had been previously treated with an anti-CD20 antibody, and wherein the patient had an NMOSD attack while being treated with the anti-CD20 antibody. In one aspect, the disclosure provides a method of treating NMOSD, the method comprising: administering the anti-CD19 antibody VIB551 to a patient in need of treatment for NMOSD, wherein the patient is an AQP4-IgG+ patient, wherein the VIB551 is administered intravenously with a first initial dose of 300 mg, a second initial dose of 300 mg two weeks after the first initial dose, and subsequent doses of 300 mg every 6 months following the first initial dose, wherein the patient had been previously treated with an anti-CD20 antibody; and, wherein the patient had an NMOSD attack within 6 months of the last dose of the anti-CD20 antibody. In aspects, the anti-CD20 antibody is rituximab (antibody C2B8 in WO94/11026).

Also provided herein are methods for treating pediatric patients with AQP4-IgG seropositive NMOSD using the anti-CD 19 antibody VIB551. NMOSD in the pediatric patients may be determined according to the criteria of Wingerchuk et al. “International consensus diagnostic criteria for neuromyelitis optica spectrum disorders. Neurology.” 2015:85(2):177-89. While the subjects are typically seropositive for AQP4-IgG, subjects having NMOSD but who are seronegative for AQP4-IgG are also contemplated. In aspects, the patient will have presented with one or more NMOSD acute relapses within the last year prior to treatment, or 2 or more NMOSD acute relapses within the last 2 years. The pediatric subjects may be male or female and may range in age from 2 to <18; for example, 2 to <6, 6 to <12, 12 to <18 or 2 to <12.

The dose administered to the pediatric patient is determined by weight. If the subject's weight is ≤37.5 kg, then 8 mg/kg IV is administered. If the patient's weight is >37.5 kg, then the subject receives 300 mg IV. Subjects who transition from ≤37.5 kg to >37.5 kg while on anti-CD19 antibody VIB551 therapy may switch regimens from 8 mg/kg to 300 mg. The anti-CD19 antibody VIB551 is typically administered as a monotherapy. Following a first dose, one or more subsequent doses may be administered. The subsequent doses may be about 24 to 28 weeks apart, about 12 months apart, about 30 months apart, about 36 months apart, or about 48 months apart, with therapy maintained as long as needed. While the anti-CD19 antibody VIB551 is typically administered as a monotherapy, corticosteroids may be co-administered during the early stages of treatment of pediatric patients. However, the corticosteroid therapy is typically tapered off shortly after antibody therapy is initiated; for example, 1 week, 2 weeks, 3 weeks or 4 weeks after the first dose is administered. In aspects, wherein the VIB551 is administered intravenously with a first initial dose of 300 mg, a second initial dose of 300 mg two weeks after the first initial dose, and subsequent doses of 300 mg every 6 months following the first initial dose.

Monoclonal antibody VIB 551 may be administered to Hepatitis B virus carriers. In Hepatitis B virus carriers who previously received anti-CD20 monoclonal antibody therapies that reduce B cell numbers, deaths due to fulminant hepatitis, aggravated hepatitis, or hepatic failure, during or after treatment, have been reported. Hepatitis B virus reactivation has also been reported in these patients. These reactions have not been reported with anti-CD 19 monoclonal antibody VIB 551, however. Nevertheless, to provide for improved patient confidence and health management, patients may have their HBV status assessed before beginning VIB 551 therapy and/or may be monitored for signs and symptoms of hepatitis B virus reactivation through periodic liver function tests and hepatitis virus marker monitoring.

Patients already on therapy with HBV infection may have their liver status monitored using parameters such as elevated or fluctuating of ALT (alanine aminotransferase)/AST (aspartate aminotransferase) ratio and a serum viral load increases. (Villadolid et al., “Hepatitis B reactivation and rituximab in the oncology practice.” Oncologist, 2010:15(10):1113-21).

Prior to administering anti-CD19 antibody VIB551 patients may be tested for HBV status. For example, using blood tests as follows. First, a patient's blood sample is prepared, serum is obtained, and HBs antigen (hereinafter HBsAg) is measured. If the HBsAg test is positive, the patient is deemed to have an active HBV infection and anti-CD19 antibody VIB551 is contraindicated. If the HBsAg test is negative, HBc antibody (hereinafter HBcAb) and/or HBs antibody (hereinafter HBsAb) are measured and if either is positive, VIB551 may be administered. Patients negative for HBsAg, HBcAb, and HBsAb do not need periodic liver function tests and hepatitis virus marker monitoring.

HBsAg, HBcAb, or HBsAb may be measured by ELISA or CLIA (including equivalent FEIA/ECLIA/CLEIA/BLEIA), using the respective antibody or antigen detection/measurement reagents. For example, HBsAg may be measured by CLEIA, using STACIA CLEIA HBs antigen (LSI Medience Corp. Japan) (www.info.pmda.go.jp/downfiles/ivd/PDF/750524_21800 AMX10883000_A_01_09.pdf) and samples with a cut-off index greater than or equal to 1 will be considered positive. HBcAb may be measured by CLIA, using ARCHITECT G06277R09 (Abbott Japan LLC; www.info.pmda.go.jp/downfiles/ivd/PDF/100159_22100AMX02283000_A_02_01.pdf) and samples with a cut-off index greater than or equal to 1 will be considered positive. For example, HBsAb may be measured by CLIA, using ARCHITECT G06255R03 (Abbott Japan LLC.) (www.info.pmda.go.jp/downfiles/ivd/PDF.100159_21000 AMY00120000_A_01_10.pdf).

In any method disclosed herein, the VIB551 may be packaged in a 10-mL vial filled with a nominal 10-mL solution of V1B551. In aspects, VIB551 may be formulated at a concentration of 10 mg/mL. In aspects, the VIB551 formulation may comprise 20 mM histidine/histidine hydrochloride, 70 mM NaCl, 106 mM (4% [w/v]) trehalose dehydrate, and 0.01% (w % v) polysorbate 80, pH 6.0.

In some embodiments, to dose of VIB551 to be administered to the patient is determined by weight. If the subject's weight is S 37.5 kg, then 8 mg/kg IV is administered. If the patient's weight is >37.5 kg, then the subject receives 300 mg IV. Subjects who transition from ≤37.5 kg to >37.5 kg while on anti-CD19 antibody VIB551 therapy may switch regimens from 8 mg/kg to 300 mg. The anti-CD19 antibody VIB551 is typically administered as a monotherapy.

The dose of VIB551 that may be used in a method of treating a patient in need of treatment for NMOSD may be a VIB551 dose that depletes at least 90% of circulating CD20+ B cells for at least six months, and does not increase risk of infections in the patient. It may be a dose of about 250 mg to about 350 mg, about 275 mg to about 325 mg, about 290 mg to about 310 mg, about 205 mg to about 305 mg, or it may be a dose of 300 mg. In aspects, the patient may receive one or more initial doses of VIB551. In one aspect, the patient may receive one, two, three or more initial doses. In particular aspects, the initial dose may be about 300 mg. In aspects, the VIB551 may be administered at an initial dose of about 250 mg to about 350 mg, about 275 mg to about 325 mg, about 290 mg to about 310 mg, about 205 mg to about 305 mg, or an initial dose of 300 mg. In particular aspects, VIB551 may be administered intravenously with a first initial dose of about 300 mg, a second initial dose of about 300 mg two weeks after the first initial dose, and subsequent doses of about 300 mg every 6 months following the first initial dose.

The dose of VIB551 that may be used in a method of treating a patient in need of treatment for NMOSD, wherein the VIB551 dose depletes at least 90% of circulating CD20+ B cells for at least six months, and does not increase risk of infections in the patient, may be a dose administered intravenously at an interval of once about every 6 months, once about every 7 months, once about every 8 months, once about every 9 months, once about every 10 months, once about every 11 months, or about once a year.

The VIB551 may also be used in a method of treating a patient in need of treatment for NMOSD in which the VIB551 is administered at a dose that (i) depletes at least 90% of circulating CD20+ B cells for at least six months, and (ii) does not increase risk of infections in the patient. The dose that depletes the at least 90% of circulating CD20+ B cells for at least six months may also deplete peripheral blood CD20− plasmablasts and plasma cells. The dose that depletes the at least 90% of circulating CD20+ B cells for at least six months may also reduce the plasma cell gene signature of the patient in need of treatment for NMOSD, or it may ablate the plasma cell gene signature of the patient in need of treatment for NMOSD. The dose that depletes the at least 90% of circulating CD20+ B cells may deplete the circulating CD20+ B cells for longer than six months. It may deplete the at least 90% of circulating CD20+ B cells for at least 9 months or at least 1 year.

The dose of VIB551 that depletes at least 90% of circulating CD20+ B cells for at least six months in the method of treating also does not increase risk of infections in the NMOSD patient. The risk of infection may not be increased in the NMOSD patient relative to his or her risk of infections prior to the administration of VIB551. The risk of infection may not be increased in the NMOSD patient in comparison to an NMOSD patient not treated with VIB551. The risk of infection may be a risk of infection by or resulting in typical pneumonia, beta haemolytic streptococcal infection, bronchitis, conjunctivis, viral conjunctivis, fungal skin infection, gastroenteritis viral, gastrointestinal infection, gingivitis, cystitis, herpes zoster, influenza, laryngitis, meningitis viral, muscle abscess, oral herpes, otitis externa, periodontitis, pneumonia, rhinitis, retinitis, pyelocystitis, retinitis, sinusitis, urinary tract infection, tinea cururis, septic shock, or upper respiratory tract infection.

Any of the methods provided herein may further comprise, prior to the administering the anti-CD19 antibody, e.g., VIB551, steps of; (i) identifying the patient as having previously been treated with the anti-CD20 antibody, (ii) determining that the patient suffered at least one NMOSD attack while being treated with the anti-CD20 antibody or had suffered at least one NMOSD attack within 6 months of the last dose of the anti-CD20 antibody, and (iii) selecting the patient for the administering the anti-CD19 antibody as a result of the determining.

Those skilled in the art will recognize, or be able to ascertain using no more than routine experimentation, many equivalents to the specific aspects described herein. Such equivalents are intended to be encompassed by the appended claims.

All publications, patents and patent applications mentioned in this specification are herein incorporated by reference into the specification to the same extent as if each individual publication, patent or patent application was specifically and individually indicated to be incorporated herein by reference.

### NUMBERED EMBODIMENTS OF THE INVENTION

Notwithstanding the appended claims, the disclosure sets forth the following numbered embodiments:
Embodiment 1. A method of treating neuromyelitis optica spectrum disorder (NMOSD), the method comprising:
administering the anti-CD19 antibody VIB551 to a patient in need of treatment for NMOSD,
wherein the patient had been previously treated with an anti-CD20 antibody, and wherein the patient had an NMOSD attack while being treated with the anti-CD20 antibody.
Embodiment 2. A method of treating NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.
Embodiment 3. The method of embodiment 1 or embodiment 2, wherein VIB551 is administered intravenously at a dose of 300 mg every 6 months.
Embodiment 4. A method of treating NMOSD, the method comprising:
administering the anti-CD19 antibody VIB551 to a patient in need of treatment for NMOSD,
wherein the patient is an astrocyte water channel aquaporin-4 (AQP4)-Immunoglobulin (Ig)G⁺ patient,
wherein the VIB551 is administered intravenously at a dose of 300 mg every 6 months,
wherein the patient had been previously treated with an anti-CD20 antibody, and wherein the patient had an NMOSD attack while being treated with the anti-CD20 antibody. Embodiment 5. A method of treating NMOSD, the method comprising:
administering the anti-CD19 antibody VIB551 to a patient in need of treatment for NMOSD,
wherein the patient is an AQP4-IgG⁺ patient,
wherein the VIB551 is administered intravenously at a dose of 300 mg every 6 months,
wherein the patient had been previously treated with an anti-CD20 antibody; and, wherein the patient had an NMOSD attack within 6 months of the last dose of the anti-CD20 antibody.
Embodiment 6. The method of any one of embodiments 3-5, wherein the patient receives at least one initial dose of VIB551.
Embodiment 7. The method of any one of embodiments 3-6, wherein the VIB551 is administered intravenously with a first initial dose of 300 mg, a second initial dose of 300 mg two weeks after the first initial dose, and subsequent doses of 300 mg every 6 months following the first initial dose.
Embodiment 8. The method of embodiment 6 or embodiment 7, wherein oral corticosteroids are co-administered to the patient with the initial VIB551 dose.
Embodiment 9. The method of embodiment 8, wherein the oral corticosteroids are administered daily for at least 2 weeks.
Embodiment 10. The method of any one of embodiments 1-9, wherein the anti-CD20 antibody is rituximab.
Embodiment 11. The method of any one of embodiments 1-10, wherein the treating is a reduction in worsening of Kurtzke Expanded Disability Severity Scale (EDSS) in the patient.
Embodiment 12. The method of embodiment 11, wherein the reduction in worsening of EDSS in the patient is:
a worsening of fewer than 2 points in EDSS score if the patient has a baseline score of 0;
a worsening of fewer than 1 point if the patient has a baseline score of 1 to 5; or
a worsening of less than 0.5 point if the patient has a baseline score of 5.5 or more.
Embodiment 13. The method of any one of embodiments 1-10, wherein the treating is a reduction in number of active magnetic resonance imaging (MRI) lesions.
Embodiment 14. The method of embodiment 13 wherein the active MRI lesions are enlarging T2 MRI lesions.
Embodiment 15. The method of any one of embodiments 1-10, wherein the treating is a reduction in number of new MRI lesions.
Embodiment 16. The method of any one of embodiments 1-10, wherein the treating is a reduction in worsening of modified Rankin Score in the patient.
Embodiment 17. The method of any one of embodiments 1-10, wherein the treating is a reduction in frequency of in-patient hospitalizations of the patient related to NMOSD.
Embodiment 18. The method of any one of embodiments 1-10, wherein the treating is a reduction of risk of an NMOSD-related attack of the patient.
Embodiment 19. The method of embodiment 18, wherein the NMOSD-related attack is characterized by appearance of anew symptom or worsening of an existing symptom related to NMOSD.
Embodiment 20. The method of embodiment 19, wherein the symptom is an eye symptom.
Embodiment 21. The method of embodiment 20, wherein the eye symptom is eye pain, blurred vision, loss of vision, or appearance of an optic nerve lesion detected by MRI.
Embodiment 22. The method of embodiment 19, wherein the symptom is a spinal cord symptom.
Embodiment 23. The method of embodiment 22, wherein the spinal cord symptom is deep or radicular pain, extremity paraesthesia, weakness, sphincter dysfunction, Lhermitte's sign, or a spinal cord lesion detectable by MRI.
Embodiment 24. The method of embodiment 19, wherein the symptom is a brain or brain stem symptom.
Embodiment 25. The method of embodiment 24, wherein the brain or brainstem symptom is nausea, double vision, oculomotor palsy, vertigo, intractable vomiting, intractable hiccups, dysarthria, dysphagia, weakness, encephalopathy, hypothalamic dysfunction, or a brain or brain stem lesion detectable by MRI.
Embodiment 26. The method of embodiment 18, wherein the reduction of risk of the NMOSD-related attack is between 60 and 85%.
Embodiment 27. The method of any one of embodiments 1-10, wherein the treating is a reduction in optic neuritis.
Embodiment 28. The method of any one of embodiments 1-10, wherein the treating is a reduction of severity of NMOSD-related attacks.
Embodiment 29. The method of embodiment 28, wherein the reduction of severity of NMOSD-related attacks is the reduction in NMOSD-related attacks graded as major.
Embodiment 30. The method of embodiment 28, wherein the reduction of severity of NMOSD-related attacks is the reduction in NMOSD attacks requiring in-patient hospitalization.
Embodiment 31. The method of any one of embodiments 1-10, wherein the treating is a decrease in NMOSD-related pain in the patient.
Embodiment 32. The method of embodiment 31, wherein the decrease in NMOSD-related pain is determined by measuring pain in legs of the patient.
Embodiment 33. The method of any one of embodiments 1-5, wherein two weeks prior to the first administering the 300 mg VIB551 every 6 months, an initial 30) mg VIB551 dose is administered to the subject.
Embodiment 34. The method of embodiment 33, wherein oral corticosteroids are co-administered to the patient with the initial 300 mg VIB551 dose.
Embodiment 35. The method of embodiment 1 or embodiment 2, wherein the patient is AQP4-IgG seropositive.
Embodiment 36. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.
Embodiment 37. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.
Embodiment 38. The method of embodiment 36 or 37, wherein the VIB551 is administered intravenously at a dose of 300 mg every 6 months.
Embodiment 39. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody, and,
wherein the VIB551 is administered intravenously at a dose of 300 mg every 6 months.
Embodiment 40. A method of reducing active MRI lesions in a patient diagnosed with NMOSD, the method comprising:
administering the anti-CD19 antibody VIB551 to a patient in need of treatment for NMOSD.
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.
Embodiment 41. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.
Embodiment 42. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.
Embodiment 43. The method of embodiment 41 or 42, wherein the VIB551 is administered intravenously at a dose of 300 mg every 6 months.
Embodiment 44. A method of reducing AQP4-IgG titers in a AQP4-IgG+ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody; and,
wherein the VIB551 is administered intravenously at a dose of 300 mg every 6 months.
Embodiment 45. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.
Embodiment 46. The method of any one of embodiments 1-45, wherein the administration depletes at least 90% of circulating CD20+ B cells for at least six months.
Embodiment 47. The method of any one of embodiments 1-46, wherein the administration does not increase risk of infections in the patient.
Embodiment 48. The method of any one of embodiments 1-47, wherein the VIB551 depletes peripheral blood CD20− plasmablasts and plasma cells within 8 days following the administering.
Embodiment 49. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.
Embodiment 50. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.
Embodiment 51. The method of embodiment 49 or 50, wherein the VIB551 is administered intravenously at a dose of 300 mg every 6 months.
Embodiment 52. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody; and,
wherein the VIB551 is administered intravenously at a dose of 300 mg every 6 months.
Embodiment 53. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.
Embodiment 54. The method of any one of embodiments 49-53, wherein the reducing the NMOSD-related disability in the patient is a reduction in a rate of worsening of NMOSD-related disability in the patient.
Embodiment 55. The method of any one of embodiments 49-54, wherein the reducing the NMOSD-related disability in the patient is a lessening of NMOSD-related disability in the patient.
Embodiment 56. The method of any one of embodiments 49-55, wherein the NMOSD-related disability is neurological disability.
Embodiment 57. The method of any one of embodiments 49-56, wherein the reducing the NMOSD-related disability is determined using EDSS.
Embodiment 58. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.
Embodiment 59. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.
Embodiment 60. The method of embodiment 58 or 59, wherein the VIB551 is administered intravenously at a dose of 300 mg every 6 months.
Embodiment 61. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody, and,
wherein the VIB551 is administered intravenously at a dose of 300 mg every 6 months.
Embodiment 62. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.
Embodiment 63. The method of embodiment 61 or embodiment 62, wherein the NMOSD-related attacks suffered by the patient comprise any one or more of an optic neuritis, a myelitis, or a brainstem attack.
Embodiment 64. The method of embodiment 63, wherein the NMOSD-related attacks suffered by the patient are clinically asymptomatic.
Embodiment 65. The method of any one of embodiments 36-64, wherein the patient receives at least one initial dose of VIB551.
Embodiment 66. The method of embodiment 65, wherein the VIB551 is administered intravenously with a first initial dose of 300 mg, a second initial dose of 300 mg two weeks after the first initial dose, and subsequent doses of 300 mg every 6 months following the first initial dose.
Embodiment 67. The method of embodiment 66, wherein oral corticosteroids are co-administered to the patient with the initial 300 mg VIB551 dose.
Embodiment 68. The method of embodiment 67, wherein the oral corticosteroids are administered daily for at least 2 weeks.
Embodiment 69. The method of any one of embodiments 36-68, wherein the anti-CD20 antibody is rituximab.
Embodiment 70. The method of any one of embodiments 1-69, wherein the VIB551 comprises a heavy chain variable region (VH) comprising the amino acid of SEQ ID NO:1 and a light chain variable region (VL) comprising the amino acid of SEQ ID NO: 2.
Embodiment 71. A method of treating NMOSD, the method comprising:
administering an anti-CD19 antibody to a patient in need of treatment for NMOSD,
wherein the patient had been previously treated with an anti-CD20 antibody, wherein the patient had an NMOSD attack while being treated with the anti-CD20 antibody.
Embodiment 72. A method of treating NMOSD, the method comprising:
administering an anti-CD19 antibody to a patient in need of treatment for NMOSD,
wherein the patient had been previously treated with an anti-CD20 antibody, wherein the patient had an NMOSD attack within 6 months of the last dose of the anti-CD20 antibody.
Embodiment 73. A method of treating NMOSD, the method comprising:
administering the anti-CD19 antibody VIB551 to a patient in need of treatment for NMOSD,
wherein the patient is an AQP4-IgG+ patient,
wherein the VIB551 is administered intravenously with a first initial dose of 300 mg, a second initial dose of 300 mg two weeks after the first initial dose, and subsequent doses of 300 mg every 6 months following the first initial dose,
wherein patient had been previously treated with an anti-CD20 antibody, and wherein the patient had an NMOSD attack while being treated with the anti-CD20 antibody.
Embodiment 74. A method of treating NMOSD, the method comprising:
administering the anti-CD19 antibody VIB551 to a patient in need of treatment for NMOSD,
wherein the patient is an AQP4-IgG⁺ patient,
wherein the VIB551 is administered intravenously with a first initial dose of 300 mg, a second initial dose of 300 mg two weeks after the first initial dose, and subsequent doses of 300 mg every 6 months following the first initial dose,
wherein the patient had been previously treated with an anti-CD20 antibody; and, wherein the patient had an NMOSD attack within 6 months of the last dose of the anti-CD20 antibody.
Embodiment 75. The method of any one of embodiments 1-74, further comprising, prior to the administering:
identifying the patient as having previously been treated with the anti-CD20 antibody; determining that the patient:
   (i) suffered at least one NMOSD attack while being treated with the anti-CD20 antibody; or
   (ii) had suffered at least one NMOSD attack within 6 months of the last dose of the anti-CD20 antibody; and
selecting the patient for the administering the anti-CD19 antibody as a result of the determining (i) or (ii).
Embodiment 76. A method of treating neuromyelitis optica spectrum disorder (NMOSD), the method comprising:
administering the anti-CD19 antibody VIB551 to a patient in need of treatment for NMOSD,
wherein the patient had been previously treated with an anti-CD20 antibody, and wherein the patient had an NMOSD attack while being treated with the anti-CD20 antibody, comprising obtaining a blood sample from the patient before administration of anti-CD19 antibody VIB551, testing for the presence of HBs antigen, HBc antibody and HBs antibody in the sample, and administering the anti-CD19 antibody VIB551 to a patient whose HBs antigen test is negative, and whose HBc antibody or HBs antibody, or both, are positive, and optionally, monitoring for periodic liver function tests and hepatitis virus marker monitoring during and after treatment with the anti-CD19 antibody VIB551.
Embodiment 77. A method of treating neuromvelitis optica spectrum disorder (NMOSD), comprising:
administering the anti-CD19 antibody VIB551 to a patient in need of treatment for NMOSD,
wherein the patient is HBs antigen-negative, and either HBs antibody-positive, HBc antibody positive, or both.
Embodiment 78. The method of embodiment 77, wherein the patient had been previously treated with an anti-CD20 antibody.
Embodiment 79. The method of embodiment 78, wherein the patient had an NMOSD attack while being treated with the anti-CD20 antibody.

### EXAMPLES

### Example 1—Rationale for Elements of Clinical Trial Design

Overview. An NMOSD clinical trial, N-MOmentum, was designed as a randomised, placebo-controlled, double-blind, 197-day, phase 2/3 study with an open-label extension period to assess the efficacy and safety of VIB551 (also referred to as VIB551 or Med1551), an anti-CD19, B-cell depleting antibody, in patients with NMOSD, recruited from 99 sites in 24 countries. Participants were randomised (3:1) using an interactive voice response system/interactive web response system, to intravenous VIB551 300 mg or placebo, respectively, administered on Days 1 and 15. Efficacy endpoints were assessed in the intent-to-treat population, and safety endpoints in the as-treated population. The primary endpoint was time to first adjudicated attack; secondary endpoints included disability worsening, magnetic resonance imaging (MRI) lesion activity, and hospitalisations. A more detailed description of the N-Momentum clinical trial can be found in Cree et al., Lancet 394: 1352-1363 (2019), and Int'l Appl. No. PCT/US20/2%13, which is incorporated herein by reference in its entirety.

Arm selection. The placebo-comparator treatment arm was chosen because there are no currently approved medications for the treatment of neuromyelitis optica spectrum disorder. The use of a placebo arm allowed for a clear and robust evaluation of VIB551, avoiding the confounding effects of other treatments, providing the highest sensitivity and robustness to detect efficacy, and helping to deliver a clinically meaningful outcome of this study.

Randomization. The 3:1 randomisation ratio used in this study was an effective and efficient approach to build an enriched safety database for VIB551 while keeping the number of required events or patients in the placebo arm at a minimum acceptable level. This randomisation ratio also addressed, to a certain degree, the ethical concerns of investigators and patients regarding enrollment of patients to a placebo arm. In addition to limiting the number of patients who received placebo, the study was designed to limit the actual duration of placebo exposure to a maximum of 197 days or time to onset of an attack, whichever occurred earlier, after which all patients had the option to enter the open-label period and to receive VIB551.

Prior to randomisation, patients were stratified based on AQP4-IgG serostatus (determined at screening) and region (Japan vs non-Japan). Within each stratum, patients were randomised in a 3:1 ratio using an interactive voice response system/interactive web response system (IVRS/IWRS) with a permuted block randomisation scheme to a treatment group and assignment of blinded investigational product kit numbers. A patient was considered randomised into the study when the investigator notified the IVRS/IWRS that the patient met eligibility criteria and the IVRS/IWRS provided the assignment of masked investigational product kit numbers to the patient.

Blinding. This was a double-blind study. VIB551 and placebo were identically labelled and indistinguishable in appearance; both were supplied as clear to opalescent, colourless to yellow liquids, and free from or practically free from particles. VIB551 and placebo doses were indistinguishable during dose preparation, handling, and infusion.

Neither the patient/legal representative nor any of the investigator or sponsor staff who were involved in the treatment or clinical evaluation of the patients were aware of the treatment received. In the event that treatment allocation for a patient became known, the sponsor was notified immediately.

Administration of the blinded dose of VIB551 or placebo on open-label period day 15 was necessary to correctly administer the loading dose of VIB551 600 mg i.v. to patients previously randomised to placebo, or to ensure that patients previously randomised to VIB551 did not receive an extra treatment dose. This blinding mechanism was implemented through the IVRS to ensure that details of the randomised treatment were not revealed to the sites.

VIB551 is known to deplete CD19+ B cells; therefore, the results of flow cytometry to count B cells were potentially unblinding. These data were not made available to investigation sites post randomisation throughout the remainder of the study.

Data from early phase development in non-oncology patient populations receiving VIB551 suggested that administration could be associated with a potential unspecified mild reduction in total immunoglobulin in individual patients. These data were not made available to investigational sites post randomisation throughout the remainder of the study because the reduction could have been potentially unblinding.

### Example 2—Clinical Trial Subject Enrollment Requirements and Criteria

Overview. Key inclusion criteria were: adults with a diagnosis of NMOSD (Wingerchuk D M, Lennon V A, Lucchinetti C F, Pittock S J, Weinshenker B G. The spectrum of neuromyelitis optica. Lancet Neurol 2007: 6: 805-15: Wingerchuk D M, Lennon V A, Pittock S J, Lucchinetti C F, Weinshenker B G. Revised diagnostic criteria for neuromyelitis optica. Neurology 2006: 66: 1485-9) and EDSS score ≤8.0, and a history of either at least one attack requiring rescue therapy (intravenous corticosteroids, intravenous immunoglobulin, and/or plasma exchange) within the year before screening or at least two attacks requiring rescue therapy in the 2 years before screening. AQP4-IgG seropositive and seronegative patients were eligible; seronegative participants had to meet the Wingerchuk 2006 criteria. Wingerchuk D M, Lennon V A, Pittock S J, Lucchinetti C F, Weinshenker B G. Revised diagnostic criteria for neuromyelitis optica. Neurology 2006: 66: 1485-9. There were no pre-planned recruitment targets with regard to AQP4-IgG serostatus. It was assumed that recruitment would reflect the known demographics of the patient population of approximately 80% seropositive, 20% seronegative. Wingerchuk D M, Lennon V A, Pittock S J, Lucchinetti C F, Weinshenker B G. Revised diagnostic criteria for neuromyelitis optica. Neurology 2006: 66: 1485-9. All participants provided written informed consent.

Sample size. The original sample size calculation concluded that 212 patients would need to be recruited to observe a required 67 attacks. This number of patients was calculated by assuming hazard rates of 1.5 per year and 1.0 per year for an attack in the placebo arm for seropositive and seronegative groups, respectively. These hazard rates were based on the observed attack rates observed in four open-label cohort studies (Bedi, et al. Impact of rituximab on relapse rate and disability in neuromyelitis optica. Mult Scler 2011: 17: 1225-30.; Costanzi, et al. Azathioprine: tolerability, efficacy, and predictors of benefit in neuromyelitis optica. Neurology 2011; 77: 659-66; Jacob, et al. Treatment of neuromyelitis optica with rituximab: retrospective analysis of 25 patients. Muscle Nerve 2008; 39: 87-90; Kim, et al. Repeated treatment with rituximab based on the assessment of peripheral circulating memory B cells in patients with relapsing neuromyelitis optica over 2 years. Arch Neurol 2011; 68: 1412-20; Pittock, et al. Eculizumab in AQP4-IgG-positive relapsing neuromyelitis optica spectrum disorders: an open-label pilot study. Lancet Neurol 2013; 12: 554-62).

The primary endpoint and the four secondary endpoints were considered to establish type 1 error control.

Primary endpoint: Time (in days) from day 1 to onset of an adjudication-committee-determined neuromyelitis optica spectrum disorder attack on or before day 197. The definition of an attack was the presence of a new symptom(s) or worsening of an existing symptom(s) related to neuromyelitis optica that met at least one of the protocol-defined criteria for a neuromyelitis optica spectrum disorder attack.

Four key secondary endpoints: 1. Worsening from baseline in EDSS score at last visit during the randomised, controlled period. The EDSS assessment in the study was performed by an independent and blinded assessor at each site using an electronic data capture system, developed by the University of Basel and Neurostatus GmBH, which contained an internal algorithm providing feedback to the assessor on inconsistencies in the EDSS assessment; 2. Change from baseline in low-contrast visual acuity binocular score measured by low-contrast Landolt C Broken Ring Chart at last visit during the randomised controlled period; 3. Cumulative total number of active MRI lesions (new gadolinium-enhancing or new/enlarging T2 lesions) during the randomised controlled period; 4. Number of neuromyelitis optica-related inpatient hospitalisations, with inpatient hospitalisation defined as more than an overnight stay.

### Example 3—Clinical Trial Protocol

Screening period. Subjects with a diagnosis of NMO/NMOSD were screened over a 28-day period to establish their eligibility to participate in the study based on the inclusion and exclusion criteria. All subjects who fulfilled eligibility criteria were randomized into the study.

Randomization. The subjects were randomized into the study in a 3:1 ratio to receive intravenously VIB551 (30 mg) or placebo as described in Table 2. Randomization occurred on Day 1 and was stratified by AQP4-IgG serostatus (in a ratio of approximately 80:20 seropositive and seronegative subjects, respectively) and by region (Japan vs non-Japan).

TABLE 2
Randomized-controlled period treatment regimen

| Treatment Arm | Treatment Regimen |
|---|---|
| 1 | 300 mg intravenous MEDI-551 on Day 1 and Day 15 |
| 2 | intravenous Placebo on Day 1 and Day 15 |

Randomized-controlled period (Day 1 to Day 197). Following randomization on Day 1, the subjects were treated with VIB551 or placebo on Day 1 and Day 15. An oral corticosteroid course was initiated on Day 1 (prednisone 20 mg/day or equivalent oral glucocorticoid) and continued until Day 14. Tapering of the oral corticosteroids occurred from Day 15 to Day 21 (for prednisone: 15 mg prednisone on Day 15, 10 mg prednisone on Day 16, 7.5 mg prednisone on Day 17, 5 mg prednisone on Days 18 and 19, and 2.5 mg prednisone on Days 20 and 21). By Day 21, tapering was completed. The rationale for use of oral corticosteroids (prednisone 20 mg/day or equivalent oral glucocorticoid) for the first 14 days (with a 1-week taper) was to provide prophylaxis against a neuromyelitis optica spectrum disorder attack for the period wherein the pharmacodynamic effect of VIB551 was not expected; a period of approximately 2-4 weeks is required for maximal B-cell depletion to occur.

During the randomized controlled period, the subjects were followed at scheduled study visits and by telephone interview. The duration of the randomized control period for each subject was planned to be 197 days. All subjects who completed the randomized control period without experiencing an NMO/NMOSD attack were given the option to enter the open-label period.

Open-label period. Subjects were given the option to enter the open label period if they: (1) completed 197 days of the randomized control period; (2) experienced an adjudication committee-determined NMO/NMOSD attack during the randomized control period; (3) were in the randomized control period at the time when 67 adjudication committee-determined NMO/NMOSD attacks had occurred; or (4) were in the randomized control period when enrollment was discontinued upon recommendation of the DMC based on evidence of efficacy and safety.

Patients who discontinued the randomised controlled period for reasons other than an adjudicated attack or the occurrence of 67 adjudicated attacks were not eligible for the open-label period. Reasons for patients not entering the open-label period were captured. These patients were then followed up for safety in the safety follow-up period.

Upon entering the open-label period for one of the four reasons outlined above, patients received VIB551 30) mg every 26 weeks; however, patients randomised to placebo during the randomised controlled period received an additional 300 mg dose on day 15 of the open-label period to maintain a total initial dose of 600 mg. Table 3 provides the open-label period treatment regimens.

TABLE 3
Open-label Period Treatment Regimen

| Treatment | |
|---|---|
| Arm in RCP | Treatment Regimen |
| 1 | 300 mg IV MEDI-551 on OLP Day 1, masked IV placebo |
| | OLP Day 15, then 300 mg IV MEDI-551 Q26W |
| | thereafter^{a} |
| 2 | 300 mg IV MEDI-551 on OLP Day 1, masked 300 mg |
| | IV MEDI-551 on OLP Day 15, then 300 mg IV |
| | MEDI-551 Q26W thereafter^{a} |

IV = intravenous; OLP = Open-label Period; Q26W = every 26 weeks; RCP = Randomized-controlled Period; SFP = Safety Follow-up Period.
^{a}The OLP will continue for a minimum of 1 year after the last subject enters and a maximum of 3 years (after the last subject enters), or until regulatory approval for MEDI-551 in the participating country, or until the Sponsor discontinues development of MED1-551 in this indication, whichever occurs first. Subjects can choose to exit the OLP at any time for any reason, including seeking alternative treatment options, at which point they will enter the SFP (unless consent is withdrawn),

During the open-label period, patients were followed up at scheduled study visits and continued to receive VIB551 therapy for a maximum of 3 years (after the last patient entered), until regulatory approval for VIB551 in each participating country or until discontinuation of the development of VIB551 by the sponsor in this indication, whichever occurred first. Patients were followed up for attacks in the same fashion as in the randomised, controlled period and events were centrally adjudicated.

Patients could choose to exit the open-label period at any time for any reason, including seeking alternative treatment options, at which point they entered the safety follow-up period (unless consent was withdrawn).

Safety follow-up period. The safety follow-up period started when a patient prematurely discontinued from the randomised controlled or open-label periods. The length of the safety follow-up period was determined by the time elapsed from the time of the last dose to the time of the premature discontinuation, to complete a total of 52 weeks. During the safety follow-up period, patients were monitored for adverse/serious adverse events, B-cell levels, anti-drug antibodies, and immunoglobulin levels. Patients could receive standard treatment for their condition, at the discretion of the investigator.

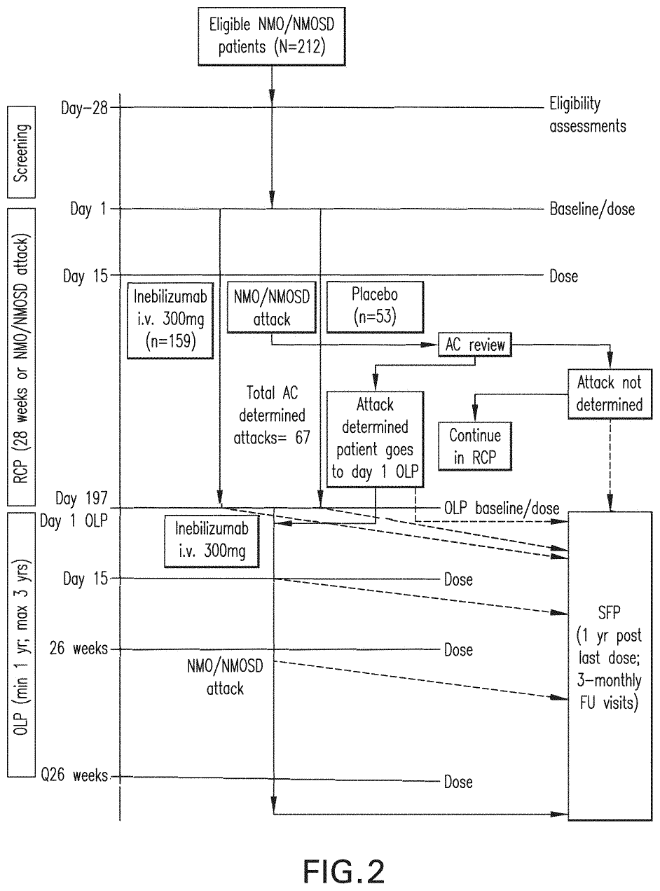

An overall study design flow diagram is provided at FIG. 2.

### Example 4—Summary of Studied Outcomes

Primary endpoint. Time (in days) from day 1 to onset of an adjudication-committee-determined neuromyelitis optica spectrum disorder attack on or before day 197. The definition of an attack was the presence of a new symptom(s) or worsening of an existing symptom(s) related to neuromyelitis optica that met at least one of the protocol-defined criteria for a neuromyelitis optica spectrum disorder attack.

Secondary endpoints. Four key secondary endpoints were considered for study-wise type 1 error control: (1) Worsening from baseline in EDSS score at last visit during the randomised, controlled period. The EDSS assessment in the study was performed by an independent and blinded assessor at each site using an electronic data capture system, developed by the University of Basel and Neurostatus GmBH, winch contained an internal algorithm providing feedback to the assessor on inconsistencies in the EDSS assessment; (2) Change from baseline in low-contrast visual acuity binocular score measured by low-contrast Landolt C Broken Ring Chart at last visit during the randomised controlled period; (3) Cumulative total number of active MRI lesions (new gadolinium-enhancing or new/enlarging T2 lesions) during the randomised controlled period; (4) Number of neuromyelitis optica-related inpatient hospitalisations, with inpatient hospitalisation defined as more than an overnight stay.

Remaining secondary endpoints. (1) Annualised attack rate (total number of adjudicated attacks, normalised by person-years) during any exposure to VIB551; (2) Treatment-emergent adverse events, including treatment-emergent serious adverse events; (3) Laboratory measurements, as well as their changes or shift from baseline over time; (4) Pharmacokinetic profile of VIB551; (5) Incidence of anti-drug antibodies directed against VIB551 for the duration of the study, both pre-dose and post-dose for each patient.

Exploratory endpoints. (1) Change from baseline in the 4-week recall 36-item Short-Form Health Survey, version 2, physical component score and mental component score at the last visit during the randomised, controlled period; (2) Change from baseline in pain numeric rating scale in five locations at the last visit during the randomised controlled period; (3) B-cell counts (total and subsets); (4) Change from baseline in plasma cell gene signature; (5) Serum AQP4-IgG titres.

### Example 5—Trial Participant Characteristics

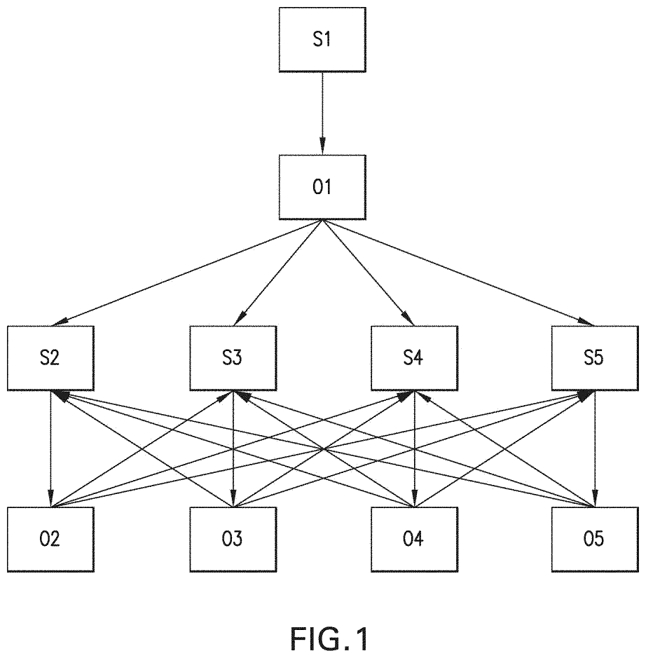

From January 2015 to October 2018, 467 participants were screened at 99 participating sites in 24 countries. Of these, 231 were enrolled, with 175 randomised to VIB551 (AQP4-IgG seropositive, n=161) and 56 to placebo (AQP4-IgG seropositive, n=52; FIG. 1). On 7 Sep. 2018, the data-monitoring committee recommended halting enrolment, owing to clear demonstration of efficacy and conditional power in excess of 99%, before the targets of 252 participants/67 adjudicated attacks were met. The sponsor discontinued enrolment on Sep. 21, 2018 before database lock and while remaining blinded to treatment assignment.

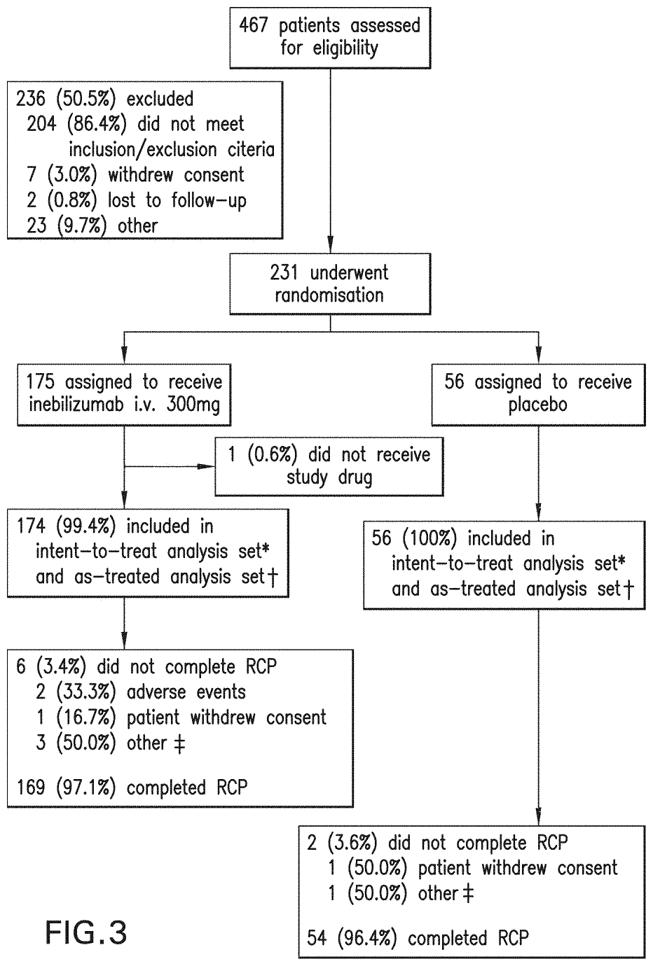

Of those assigned to VIB551, 174 (99-4%) were included in analysis populations (one participant [0.6%] did not receive study drug); 169 (97-1%) participants completed the randomised, controlled period, six discontinued owing to adverse events (n=2), withdrawn consent (n=1), or ‘other’ (n=3). All 56 participants assigned to placebo received intervention and were included in the analyses, with 54 (96.4%) completing the randomised, controlled period; two participants discontinued (n=1 withdrawn consent and n=1 ‘other’; FIG. 3). Most participants were women (n=209, 90.9%; table 4) and white (n=120, 52-2%, table 4). Participant demographics were broadly similar between treatment groups in the overall and AQP4-IgG seropositive populations (table 4). The open-label period is ongoing, with 213 participants receiving VIB551 (original randomisation: VIB551, n=162; placebo, n=51).

TABLE 4
Patient demographic and baseline characteristics

| | AQP4-IgG seropositive; | | | | Overall ITT population; | | | |
|---|---|---|---|---|---|---|---|---|
| | n = 213 | | | | n = 230 | | | |
| | Placebo | | VIB551 | | Placebo | | VIB551 | |
| Demographic/characteristic | n = 52 | | n = 161 | | n = 56 | | n = 174 | |
| Age | | | | | | | | |
| Mean (SD), years | 42.4 | (14.3) | 43.2 | (11.6) | 42.6 | (13.9) | 43.0 | (11.6) |
| Sex | | | | | | | | |
| Women | 49 | (94.2) | 151 | (93.8) | 50 | (89.3) | 159 | (91.4) |
| Race | | | | | | | | |
| American Indian or Alaskan Native | 5 | (9.6) | 11 | (6.8) | 5 | (8.9) | 14 | (8.0) |
| Asian | 8 | (15.4) | 37 | (23.0) | 8 | (14.3) | 39 | (22.4) |
| Black or African American | 5 | (9.6) | 14 | (8.7) | 5 | (8.9) | 15 | (8.6) |
| White | 24 | (46.2) | 86 | (53.4) | 28 | (50.0) | 92 | (52.9) |
| Other | 10 | (19.2) | 12 | (7.5) | 10 | (17.9) | 13 | (7.5) |
| Multiple categories checked | 0 | | 1 | (0.6) | 0 | | 1 | (0.6) |
| Ethnicity | | | | | | | | |
| Hispanic or Latino | 15 | (28.8) | 25 | (15.5) | 15 | (26.8) | 28 | (16.1) |
| Disease duration, years | | | | | | | | |
| Mean (SD) | 2.92 | (3.54) | 2.49 | (3.39) | 2.77 | (3.45) | 2.41 | (3.30) |
| Duration ≥5 years | 10 | (19.2) | 29 | (18.0) | 10 | (17.9) | 30 | (17.2) |
| Time since first relapse, years | | | | | | | | |
| Mean (SD) | 5.19 | (5.69) | 5.19 | (5.90) | 4.88 | (5.59) | 5.12 | (5.79) |
| Number of prior relapses | | | | | | | | |
| ≥2 relapses | 39 | (75.0) | 137 | (85.1) | 42 | (75.0) | 149 | (85.6) |
| Type of most recent attack | | | | | | | | |
| Optic neuritis | 19 | (36.5) | 77 | (47.8) | 21 | (37.5) | 85 | (48.9) |
| Myelitis | 32 | (61.5) | 94 | (58.4) | 34 | (60.7) | 99 | (56.9) |
| Brain/brainstem | 8 | (15.4) | 6 | (3.7) | 10 | (17.9) | 8 | (4.6) |
| Baseline AAR | | | | | | | | |
| Mean (SD) | 1.46 | (1.36) | 1.68 | (1.49) | 1.57 | (1.46) | 1.73 | (1.53) |
| Prior treatment | | | | | | | | |
| Any therapy* | 51 | (98.1) | 159 | (98.8) | 55 | (98.2) | 172 | (98.9) |
| Plasmapheresis | 26 | (50.0) | 58 | (36.0) | 27 | (48.2) | 67 | (38.5) |
| Intravenous immunoglobulin | 3 | (5.8) | 8 | (5.0) | 3 | (5.4) | 8 | (4.6) |
| Any prior maintenance therapy | 36 | (69.2) | 108 | (67.1) | 38 | (67.9) | 114 | (65.5) |
| Corticosteroids | 21 | (40.4) | 74 | (46.0) | 23 | (41.1) | 79 | (45.4) |
| Non-biologic immunosuppression† | 25 | (48.1) | 77 | (47.8) | 26 | (46.4) | 79 | (45.4) |
| Azathioprine | 21 | (40.4) | 62 | (38.5) | 22 | (39.3) | 63 | (36.2) |
| Mycophenolate mofetil | 7 | (13 5) | 25 | (15.5) | 7 | (12.5) | 26 | (14.9) |
| Methotrexate | 0 | | 2 | (1.2) | 0 | | 2 | (1.1) |
| Biologic agent | 5 | (9.6) | 23 | (14.3) | 5 | (8.9) | 25 | (14.4) |
| Rituximab | 4 | (7.7) | 12 | (7.5) | 4 | (7.1) | 13 | (7.5) |
| Interferon beta | 1 | (1 9) | 6 | (3.7) | 1 | (1.8) | 7 | (4.0) |
| Natalizumab | 0 | | 2 | (1.2) | 0 | | 2 | (11) |
| Glatiramer acetate | 0 | | 2 | (1.2) | 0 | | 2 | (11) |
| No prior maintenance therapy | 16 | (30.8) | 53 | (32.9) | 18 | (32.1) | 60 | (34.5) |
| Baseline Gd-enhancing lesions | | | | | | | | |
| Mean (SD) | 0.8 | (0.9) | 1.2 | (1.2) | 0.9 | (0.9) | 1.2 | (1.2) |
| Median (range) | 1.0 | (0.0-4-0) | 1.0 | (0.0-5.0) | 1.0 | (0.0-4.0) | 1.0 | (0.0-5.0) |
| Baseline EDSS score | | | | | | | | |
| Mean (SD) | 4.35 | (1.63) | 3.81 | (1.77) | 4.19 | (1.68) | 3.81 | (1.81) |
| Median (range) | 4.0 | (1.0-8.0) | 3.5 | (0.0-8.0) | 4.0 | (1.0-8.0) | 3.5 | (0.0-8.0) |
| Weight, kg | n = 52 | | n = 160 | | n = 56 | | n = 173 | |
| Mean (SD) | 71.79 | (19.97) | 68.16 | (17.55) | 71.61 | (19.26) | 68.37 | (17-42) |
| Height, cm | n = 52 | | n = 159 | | n = 56 | | n = 172 | |
| Mean (SD) | 161.81 | (7.25) | 163.86 | (7.73) | 162.55 | (7.73) | 164.30 | (7.97) |
| BMI, kg/m² | n = 52 | | n = 159 | | n = 56 | | n = 172 | |
| Mean (SD) | 27.30 | (6.90) | 25.29 | (5.64) | 27.02 | (6.73) | 25.22 | (5.51) |
| BMI category | n = 52 | | n = 159 | | n = 56 | | n = 172 | |
| Underweight (<18.5 kg/m²) | 1 | (1.9) | 10 | (6.3) | 1 | (1.8) | 10 | (5.8) |
| Normal (18.5-<25.0 kg/m²) | 25 | (48.1) | 79 | (49.7) | 28 | (50.0) | 85 | (49.4) |
| Overweight (25.0-<30.0 kg/m²) | 10 | (19.2) | 45 | (28.3) | 11 | (19.6) | 52 | (30.2) |

TABLE 5
AC-determined NMOSD attacks by recovery grades, randomized control period
(intent-to-treat population)

| | AQP4-1gG sero+ | | AQP4-IgG sero− | | Total | |
|---|---|---|---|---|---|---|
| | N = 213 | | N = 17 | | N = 230 | |
| | Placebo | Inebilizumab | Placebo | Inebilizumab | Placebo | Inebilizumab |
| | N = 52 | N = 161 | N = 4 | N = 13 | N = 56 | N = 174 |
| AC-determined | 22 | 18 (11.2%) | 0 | 3 (23.1%) | 22 (39.3%) | 21 (12.1%) |
| attack | (42.3%) | | | | | |
| Attack | | | | | | |
| recovery grade | | | | | | |
| Major | 2 (9.1%) | 2 (11.1%) | 0 | 0 | 2 (9.1%) ^{a} | 2 (9.5%) ^{b} |
| Minor | 6 | 5 (27.8%) | 0 | 0 | 6 (27.3%) ^{a} | 5 (23.8%) ^{b} |
| | (27.3%) | | | | | |
| No recovery | 9 | 4 (22.%) | 0 | 2 (66.7%) | 9 (40.9%) ^{a} | 6 (28.6%) ^{b} |
| | (40.9%) | | | | | |

AC = Adjudication Committee; AQP4-IgG = autoantibodies against aquaporin-4; ITT = intent-to-treat; NMOSD = neuromyelitis optica spectrum disorders; ON = optic neuritis; RCP = randomized-controlled period; sero+ = seropositive; sero− = seronegative.
^{a} In the placebo group, the number of subjects for whom recovery data was collected was 17. Using this as a denominator to calculate percentages yields the following: Major, 11.8%; Minor, 35.5%; No recovery, 52.9%.
^{b} In the inebilizumab group, the number of subjects for whom recovery data was collected was 13. Using this as a denominator to calculate percentages yields the following: Major, 15.4%; Minor, 38.5%; No recovery, 46.2%.

An annualized attack rate (total number of AC-determined NMOSD attacks normalized by person years) during any exposure to VIB551 was also determined. Of note, an annualized attack rate could not also be calculated for the placebo treatment period because subjects were removed from the placebo-controlled portion of the study after an AC-adjudicated attack. Accordingly, any such calculation in the placebo period would have been biased and would have potentially overestimated the attack rate. Subjects in the VIB551 treatment group, however, remained in the study receiving VIB551 following an attack, and therefore, an estimate of annualized attack rate could be calculated for the period in which subjects were treated with VIB551.

The annualized AC-determined NMOSD attack rate in any subject treated with VIB551 was low, at 0.126. See Table 6. When calculated separately for the AQP4-IgG seropositive and AQP4-IgG seronegative subjects, the annualized attack rate was 0.13 and 0.088, respectively.

TABLE 6
Annualized Adjudication Committee-determined
NMOSD Attack Rate (Any VIB551 Population)

| | AQP4-IgG sero+ | AQP4-IgG sero− | Total |
|---|---|---|---|
| | N = 208 | N = 17 | N = 225 |
| Number of AC- | 42 | 3 | 45 |
| determined attack | | | |
| Total person-year ^{a} | 323.595 | 34.152 | 357.747 |
| Annualized attack rate ^{b} | 013 | 0.088 | 0.126 |

AC = Adjudication Committee; AQP4-IgG = autoantibodies against aquaporin-4; SFP = safety follow-up period; sero− = seronegative; sero+ = seropositive.
^{a} Total person-years will be calculated as the sum of the person-years for individual subject. Person-year for individual subject is defined as (Date of last day before SFP − 1st inebilizumab dose date + 1)/365.25.
^{b} Annualized attack rate is defined as total number of AC-determined attacks divided by total person-years.

The study was stopped early on the recommendation of the independent data-monitoring committee owing to the clear demonstration of efficacy.

TABLE 7
Key secondary outcomes

| | AQP4-IgG seropositive; n = 213 | | | Overall ITT population; n = 230 | | |
|---|---|---|---|---|---|---|
| | Placebo | VIB551 | | Placebo | VIB551 | |
| Secondary endpoint | n = 52 | n = 161 | p value* | n = 56 | n = 174 | p value* |
| Worsening from baseline in | n = 52 | n = 161 | | n = 56 | n = 174 | |
| EDSS score at last visit^{†} | | | | | | |
| n (%) | 18 (34.6) | 25 (15.5) | | 19 (33.9) | 27 (15.5) | |
| OR (95% CI) | 0.371 (0.181-0.763) | | 0.021 | 0.370 (0.185-0.739) | | 0.021 |
| Change from baseline in | n = 52 | n = 158 | | n = 56 | n = 171 | |
| LCVAB score^{‡} | | | | | | |
| LSM (SE) | 0.600 (0.999) | 0.562 (0.572) | | 1.442 (1.217) | 1.576 (0.935) | |
| LSM difference (95% | −0.038 (−2.312-2.236) | | 0.974 | 0.134 (−2.025-2.294) | | 0.974 |
| CI) | | | | | | |
| Cumulative number of active | n = 31 | n = 74 | | n = 32 | n = 79 | |
| MRI lesions^{§} | | | | | | |
| Mean (SD) | 2.3 (1.3) | 1.7 (1.0) | | 2.3 (1.3) | 1.6 (1.0) | |
| RR (95% CI)^{¶} | 0.568 (0.385-0.836) | | 0.017 | 0.566 (0.387-0·828) | | 0.017 |
| Cumulative number of | n = 7 | n = 8 | | n = 8 | n = 10 | |
| inpatient hospitalisations∥ | | | | | | |
| Mean (SD) | 1.4 (0.8) | 1.0 (0.0) | | 1.4 (0.7) | 1.0 (0.0) | |

| | | | | |
|---|---|---|---|---|
| RR (95% CI)^{¶} | 0.258 (0.090-0.738) | 0.023 | 0.286 (0.111-0.741) | 0.023 |

*Presented p values are adjusted for multiple comparison testing; differences were considered significant if p <0.05.
^{†}Proportion of participants with worsening EDSS score from baseline, with OR calculated using a logistic regression model with treatment, serostatus, and baseline score as explanatory variables and non-responder imputation (with missing values considered as worsening).
^{‡}LSM differences in the change in LCVAB score were assessed using an analysis of covariance model with treatment, serostatus, and baseline Landolt C Broken Ring Chart binocular score as explanatory variables and last non-missing low-contrast visual acuity score.
^{§}Cumulative number of active MRI lesions from baseline (includes gadolinium-enhancing or new/enlarging T2 lesions), with RRs assessed using negative binomial regression, with treatment and serostatus as explanatory variables
^{¶}RR analysis is based on the entire population, not just those who had an event.
∥Cumulative number of neuromyelitis optica-related inpatient hospitalisations from baseline, with RRs assessed using negative binomial regression, with treatment and serostatus as explanatory variables. AQP4-IgG, 161 aquaporin-4-immunoglobulin G; CI, confidence interval; EDSS, Expanded Disability Status Scale; ITT, intent-to-treat; LCVAB, low-contrast visual acuity binocular; LSM, least-squares mean; MRI, magnetic resonance imaging; OR, odds ratio; RR, rate ratio; SD, standard deviation; SE, standard error.

### Example 6: Safety and Efficacy of VIB551 in Those with Previous Rituximab Exposure

Seventeen subjects enrolled in N-MOmentum (7.4%) had previous rituximab treatment. Demographics and baseline characteristics of participants in N-MOmentum with prior rituximab use are shown in Table 8, including that median time between the last rituximab use and randomization was 1.5 years. Baseline characteristics of participants with prior use of rituximab were similar to those without prior experience with rituximab (n=208).

TABLE 8
Characteristics of Participants in
N-MOmentum with Prior Rituximab Use

| | Randomized control group | | | | | |
|---|---|---|---|---|---|---|
| | Inebilizumab | | Placebo | | Overall | |
| Parameter | (n = 13) | | (n = 4) | | (n-17) | |
| Age, median (IQR), y | 47 | (32-50) | 38 | (29-46) | 46 | (31-49) |
| Women, n (%) | 13 | (100) | 3 | (75) | 16 | (94) |
| White or Asian, n (%) | 4 | (31) | 1 | (25) | 5 | (29) |
| AQP-IgG seropositive | 12 | (92) | 4 | (100) | 16 | (94) |
| B cell count (IPR) | 342.6 | | 198.9 | | 308.7 | |
| | (181.2-319.3) | | (67.8-330.1) | | (171.8-319.3) | |
| Time between last | 1.5 | (0.8-4.4) | 1.3 | (0.9-3.1) | 1.5 | (0.8-4.4) |
| rituximab dose and | | | | | | |
| first dose | | | | | | |
| inebilizumab, | | | | | | |
| median (range), y | | | | | | |
| Rituximab doses, | 1 | (1-11) | 1 | (1-2) | 1 | (1-11) |
| median (range), n | | | | | | |
| Attack while on | 5 | (38) | 2 | (50) | 7 | (41) |
| rituximab, n (%) | | | | | | |

| | | | |
|---|---|---|---|
| AAR before first dose | 0.728 | 0.924 | 0.779 |
| of inebilizumab | (0.342-1.886) | (0.401-1.875) | (0.342-1.886) |
| (range) | | | |

AQP4, aquaporin-4;
AAR, annualized attack rate,
IgG, immunoglobulin G;
IQR, interquartile range

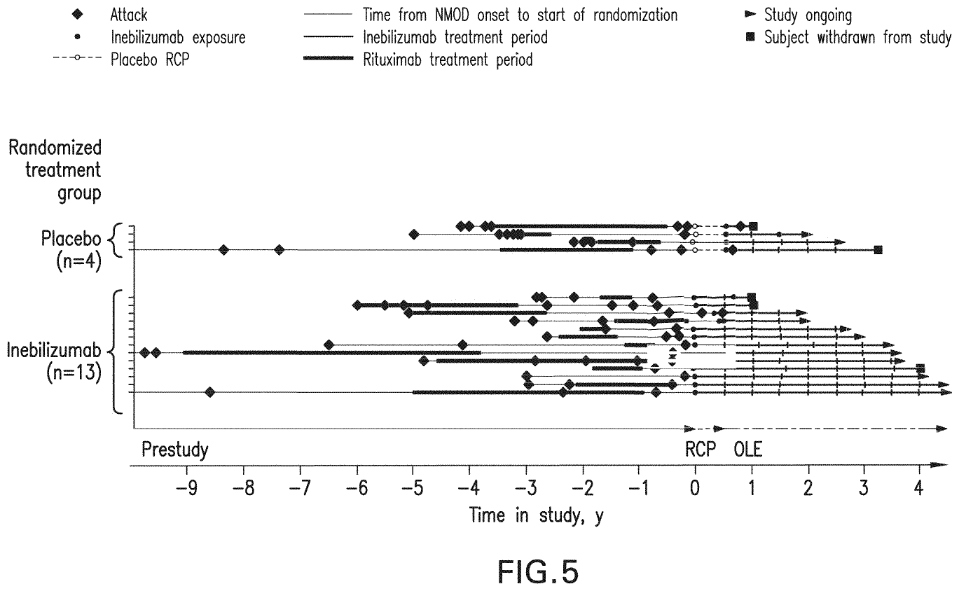

A summary of the attack history for the each of the 17 N-MOmentum participants with prior rituxumab usage is presented in FIG. 5. Three of the 17 participants had attacks following exposure to inebilizumab. Of the three attacks, one occurred during the RCP of a participant randomized into the inebilizumab group and two occurred during the OLP of participants randomized into the placebo group. The annualized attack rate (AAR) prior to and after inebilizumab administration was 0.78 (median: 1.08), and 0.11 (95% CI 0.02-0.33), respectively; and the AAR for those with and without prior rituximab exposure was 0.083 and 0.102, respectively. The three rituximab-experienced participants who had attacks while taking inebilizumab had 1 attack each during their time in the study (1.54 years for the participant randomized to inebilizumab; 0.51 and 2.74 years for the participants randomized to placebo). All 3 attacks were myelitis, one of which was graded as mild by the opticospinal impairment scale.

Seven of 17 patients entered the study as rituximab ‘failures’, defined as having an NMOSD attack while on (or within 6 months) of the last dose of rituximab. None of the 7 failures had an adjudicated attack after receiving inebilizumab (mean follow-up 2.6 years).

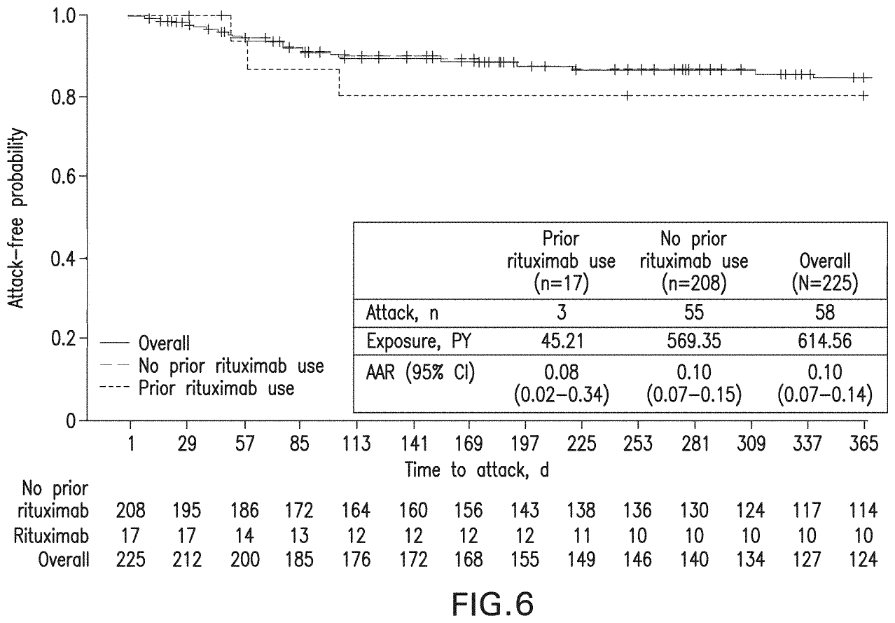

Attack-free probability stratified by prior rituximab use is provided in FIG. 6. After the first inebilizumab administration, the AAR of rituximab-experienced participants was similar to those without prior rituximab use (0.083 and 0.102 attacks/person-year, respectively). Secondary endpoints, such as change from baseline in EDSS scores, number of active lesions on MRI, and number of NMOSD-related hospital stays were also assessed. See Table 9. Among 13 participants with prior use of rituximab who received inebilizumab during the randomized control period, 2 (15%) experienced worsening of EDSS scores, 6 (46%) had active lesions on MRI, and 1 (8%) had a hospital stay related to NMOSD. One of the 2 cases of EDSS score worsening and hospitalization were related to the attack that occurred during the randomized controlled period. Secondary outcomes in the inebilizumab groups were generally similar regardless of prior rituximab use.

TABLE 9
Secondary Endpoints During the Randomized
Control Period, by Prior Use of Rituximab

| | Randomized to inebilizumab | | |
|---|---|---|---|
| | Prior | No prior use | |
| | rituximab use | of rituximab | Placebo |
| Endpoint | (n = 13) | (n = 161) | (n = 56)^{a} |
| EDSS, worsening from | 2 (15) | 37 (21) | 19 (34) |
| baseline, n (%) | | | |
| MRI lesion,^{b} n (%) | 6 (46) | 73 (45) | 32 (57) |
| Lesion per participant,^{c} | 2.2 (1.6) | 1.6 (1.0) | 2.3 (1.3) |
| mean (SD) | | | |
| Hospital stay related to | 1 (8) | 9 (6) | 8 (14) |
| NMOSD,^{d} n (%) | | | |
| Stay per participant^{e} mean | 1 | 1 | 1.4 (0.7) |
| (SD) | | | |

EDSS, Expanded Disability Status Scale; MRI, magnetic resonance imaging; NMOSD, neuromyelitis optica spectrum disorder; SD, standard deviation.
^{a}Includes all participant randomized to the placebo group, regardless of prior rituximab use.
^{b}Gadolinium-enhancing or new or enlarging T2 lesions measured across the optic nerve, brain, brainstem, and spinal cord ¹¹
^{c}Among participants with lesions on MRI.
^{d}Longer than an overnight hospital stay. ¹¹
^{e}Among participants with hospital stays.

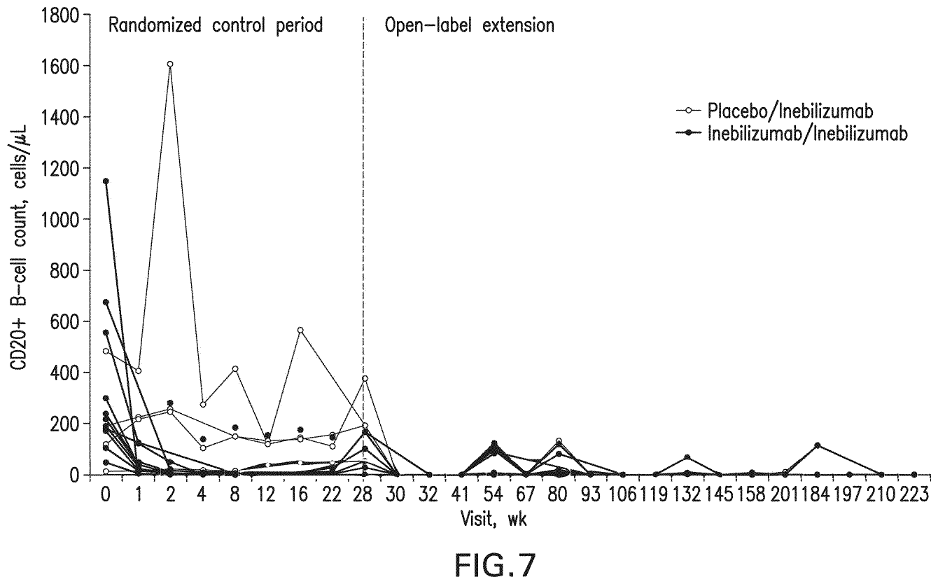

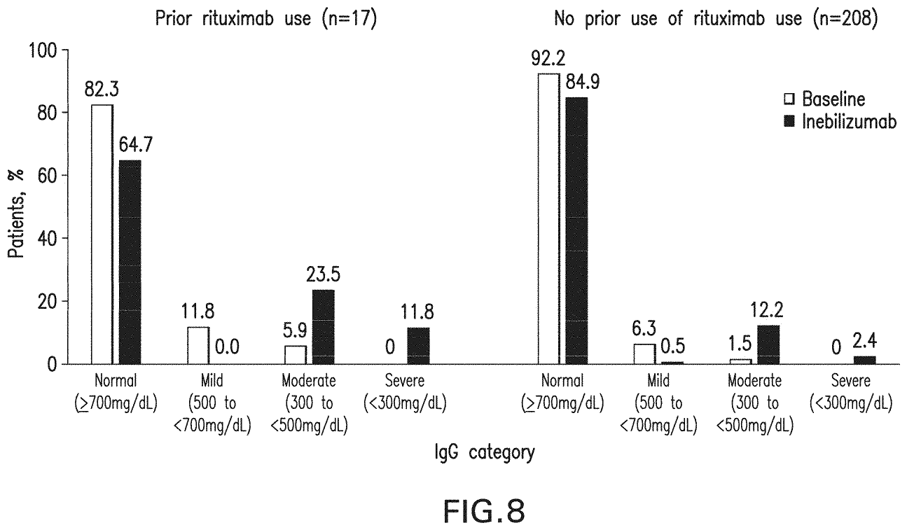

B-cell depletion kinetics in participants previously treated with rituximab was also examined and was similar to that of the general inebilizumab-treated study population. See FIG. 7. After inebilizumab treatment, participants with and without prior rituximab use experienced annual reductions from baseline in IgG levels of 42.3 mg/dL/year and 49.5 mg/dL/year, respectively (P 0.6687). While they were receiving inebilizumab, 6 (35%) participants with prior rituximab use and 30 (15%) participants without prior rituximab use experienced IgG levels <500 mg/dL (FIG. 8). No correlation between IgG level and infections was observed in a previous analysis of the N-MOmentum study. Generally similar lymphocyte and neutrophil counts by toxicity grade were observed with the inebilizumab treatment group regardless of prior rituximab experience, with most reported as grade 0/1 in both groups.

Adverse events of interest in this group included infusion reaction (2), infections (16), and cytopenia (1). Table 10 provides a summary of treatment emergent adverse events of subjects having had prior, versus those not having had prior, rituximab use.

Nine of 17 (53%) participants with prior rituximab use and 79 of 208 (38%) without prior rituximab use experienced a TEAE associated with inebilizumab (Table 10). Low rates were observed of serious TEAEs related to inebilizumab in both groups. Serious TEAEs related to inebilizumab were reported in 2 (12%) participants with prior rituximab use and included urinary tract infection and cellulitis. The proportions of participants who experienced infusion-related reactions while receiving inebilizumab were similar regardless of prior rituximab use. Most participants, with or without prior rituximab experience (94% and 70%, respectively), had ≥1 infection while receiving inebilizumab. Serious and grade ≥3 infections occurred in 3 (18%) participants of those with prior rituximab experience and 20 (10%) participants of those without prior rituximab experience. In participants with prior rituximab use, serious and/or grade ≥3 infections included nasophayngitis (grade 3) urinary tract infection (serious and grade 3), cellulitis (serious and grade 3), and perforated appendicitis (serious and grade 4), all occurring in participant each. No deaths, opportunistic infections, or cases of progressive multifocal leukoencephalopathy, a recognized complication of B-cell-depleting therapies, were reported among the 17 rituximab-experienced participants. The most common AEs in participants with prior rituximab use were urinary tract infection and influenza.

TABLE 10
TEAEs, Serious TEAEs, and TEAES of Special
Interest After Receiving Inebilizumab

| | | |
|---|---|---|
| | | No prior use |
| | Prior rituximab | of rituximab |
| Event n (%) | use (n = 17) | (n = 208) |

TEAE

| | | | | |
|---|---|---|---|---|
| Any | 17 | (100) | 190 | (91) |
| Related to inebilizumab | 9 | (53) | 79 | (38) |
| Leading to treatment | 1 | (6) | 6 | (3) |
| discontinuation | | | | |
| Grade ≥3 | 5 | (29) | 46 | (22) |
| Serious | 6 | (35) | 38 | (18) |
| Serious and related to inebilizumab | 2 | (12) | 9 | (4) |

| | | | |
|---|---|---|---|
| Death | 0 | 2 | (1) |

TEAE of special interest

| Any | 16 | (94) | 157 | (76) |
|---|---|---|---|---|
| Infusion-related reaction | 2 | (12) | 25 | (12) |
| Anaphylactic reaction | 0 | | 0 | |
| Hypersensitivity | 1 | (6) | 2 | (1) |
| Infections | 16 | (94) | 146 | (70) |
| Serious | 3 | (18) | 20 | (10) |
| Grade 1 | 9 | (53) | 115 | (55) |
| Grade 2 | 12 | (71) | 75 | (36) |
| Grade 3 | 2 | (12) | 17 | (8) |
| Grade 4 | 1 | (6) | 4 | (2) |
| Grade 5 | 0 | | 1 | (<1) |
| Hepatic function abnormality | 1 | (6) | 14 | (7) |
| Cytopenia | 1 | (6) | 12 | (6) |

| | | | |
|---|---|---|---|
| Opportunistic infections | 0 | 2 | (1) |

| | | |
|---|---|---|
| Confirmed PML | 0 | 0 |

PML, progressive multifocal leukoencephalopathy; TEAE, treatment-emergent adverse event.

1. A method of treating neuromyelitis optica spectrum disorder (NMOSD), the method comprising:
administering the anti-CD19 antibody VIB551 to a patient in need of treatment for NMOSD,
wherein the patient had been previously treated with an anti-CD20 antibody.

2. A method of treating NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with or within 6 months of the last dose of the anti-CD20 antibody.

3. The method of claim 1 or claim 2, wherein VIB551 is administered intravenously at a dose of 300 mg every 6 months.

4. A method of treating NMOSD, the method comprising:
administering the anti-CD19 antibody VIB551 to a patient in need of treatment for NMOSD,
wherein the patient is an astrocyte water channel aquaporin-4 (AQP4)-Immunoglobulin (Ig)G⁺ patient,
wherein the VIB551 is administered intravenously at a dose of 300 mg every 6 months,
wherein the patient had been previously treated with an anti-CD20 antibody, and wherein the patient had an NMOSD attack while being treated with the anti-CD20 antibody.

5. A method of treating NMOSD, the method comprising:
administering the anti-CD19 antibody VIB551 to a patient in need of treatment for NMOSD,
wherein the patient is an AQP4-IgG⁺ patient,
wherein the VIB551 is administered intravenously at a dose of 300 mg every 6 months,
wherein the patient had been previously treated with an anti-CD20 antibody; and, wherein the patient had an NMOSD attack within 6 months of the last dose of the anti-CD20 antibody.

6. The method of any one of claims 3-5, wherein the patient receives at least one initial dose of VIB551.

7. The method of any one of claims 3-6, wherein the VIB551 is administered intravenously with a first initial dose of 300 mg, a second initial dose of 300 mg two weeks after the first initial dose, and subsequent doses of 300 mg every 6 months following the first initial dose.

8. The method of claim 6 or claim 7, wherein oral corticosteroids are co-administered to the patient with the initial VIB551 dose.

9. The method of claim 8, wherein the oral corticosteroids are administered daily for at least 2 weeks.

10. The method of any one of claims 1-9, wherein the anti-CD20 antibody is rituximab.

11. The method of any one of claims 1-10, wherein the treating is a reduction in worsening of Kurtzke Expanded Disability Severity Scale (EDSS) in the patient.

12. The method of claim 11, wherein the reduction in worsening of EDSS in the patient is:
a worsening of fewer than 2 points in EDSS score if the patient has a baseline score of 0;
a worsening of fewer than 1 point if the patient has a baseline score of 1 to 5: or
a worsening of less than 0.5 point if the patient has a baseline score of 5.5 or more.

13. The method of any one of claims 1-10, wherein the treating is a reduction in number of active magnetic resonance imaging (MRI) lesions.

14. The method of claim 13 wherein the active MRI lesions are enlarging T2 MRI lesions.

15. The method of any one of claims 1-10, wherein the treating is a reduction in number of new MRI lesions.

16. The method of any one of claims 1-10, wherein the treating is a reduction in worsening of modified Rankin Score in the patient.

17. The method of any one of claims 1-10, wherein the treating is a reduction in frequency of in-patient hospitalizations of the patient related to NMOSD.

18. The method of any one of claims 1-10, wherein the treating is a reduction of risk of an NMOSD-related attack of the patient.

19. The method of claim 18, wherein the NMOSD-related attack is characterized by appearance of a new symptom or worsening of an existing symptom related to NMOSD.

20. The method of claim 19, wherein the symptom is an eye symptom.

21. The method of claim 20, wherein the eye symptom is eye pain, blurred vision, loss of vision, or appearance of an optic nerve lesion detected by MRI.

22. The method of claim 19, wherein the symptom is a spinal cord symptom.

23. The method of claim 22, wherein the spinal cord symptom is deep or radicular pain, extremity paraesthesia, weakness, sphincter dysfunction, Lhermitte's sign, or a spinal cord lesion detectable by MRI.

24. The method of claim 19, wherein the symptom is a brain or brain stem symptom.

25. The method of claim 24, wherein the brain or brainstem symptom is nausea, double vision, oculomotor palsy, vertigo, intractable vomiting, intractable hiccups, dysarthria, dysphagia, weakness, encephalopathy, hypothalamic dysfunction, or a brain or brain stem lesion detectable by MRI.

26. The method of claim 18, wherein the reduction of risk of the NMOSD-related attack is between 60 and 85%.

27. The method of any one of claims 1-10, wherein the treating is a reduction in optic neuritis.

28. The method of any one of claims 1-10, wherein the treating is a reduction of severity of NMOSD-related attacks.

29. The method of claim 28, wherein the reduction of severity of NMOSD-related attacks is the reduction in NMOSD-related attacks graded as major.

30. The method of claim 28, wherein the reduction of severity of NMOSD-related attacks is the reduction in NMOSD attacks requiring in-patient hospitalization.

31. The method of any one of claims 1-10, wherein the treating is a decrease in NMOSD-related pain in the patient.

32. The method of claim 31, wherein the decrease in NMOSD-related pain is determined by measuring pain in legs of the patient.

33. The method of any one of claims 1-5, wherein two weeks prior to the first administering the 300 mg VIB551 every 6 months, an initial 300 mg VIB551 dose is administered to the subject.

34. The method of claim 33, wherein oral corticosteroids are co-administered to the patient with the initial 300 mg VIB551 dose.

35. The method of claim 1 or claim 2, wherein the patient is AQP4-IgG seropositive.

36. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.

37. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.

38. The method of claim 36 or 37, wherein the VIB551 is administered intravenously at a dose of 300 mg every 6 months.

39. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody, and,
wherein the VIB551 is administered intravenously at a dose of 300 mg every 6 months.

40. A method of reducing active MRI lesions in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.

41. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.

42. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.

43. The method of claim 41 or 42, wherein the VIB551 is administered intravenously at a dose of 300 mg every 6 months.

44. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody; and,
wherein the VIB551 is administered intravenously at a dose of 300 mg every 6 months.

45. A method of reducing AQP4-IgG titers in a AQP4-IgG⁺ patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.

46. The method of any one of claims 1-45, wherein the administration depletes at least 90% of circulating CD20+ B cells for at least six months.

47. The method of any one of claims 1-46, wherein the administration does not increase risk of infections in the patient.

48. The method of any one of claims 1-47, wherein the VIB551 depletes peripheral blood CD20⁻ plasmablasts and plasma cells within 8 days following the administering.

49. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.

50. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.

51. The method of claim 49 or 50, wherein the VIB551 is administered intravenously at a dose of 300 mg every 6 months.

52. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody; and,
wherein the VIB551 is administered intravenously at a dose of 300 mg every 6 months.

53. A method of reducing NMOSD-related disability in a patient diagnosed with NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.

54. The method of any one of claims 49-53, wherein the reducing the NMOSD-related disability in the patient is a reduction in a rate of worsening of NMOSD-related disability in the patient.

55. The method of any one of claims 49-54, wherein the reducing the NMOSD-related disability in the patient is a lessening of NMOSD-related disability in the patient.

56. The method of any one of claims 49-55, wherein the NMOSD-related disability is neurological disability.

57. The method of any one of claims 49-56, wherein the reducing the NMOSD-related disability is determined using EDSS.

58. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody.

59. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody.

60. The method of claim 58 or 59, wherein the VIB551 is administered intravenously at a dose of 300 mg every 6 months.

61. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack while being treated with the anti-CD20 antibody, and,
wherein the VIB551 is administered intravenously at a dose of 300 mg every 6 months.

62. A method of reducing NMOSD-related attacks in a patient in need of treatment for NMOSD, the method comprising:
administering the anti-CD19 antibody VIB551 to a patient in need of treatment for NMOSD,
wherein the patient had been previously treated with an anti-CD20 antibody, wherein the patient had an NMOSD attack within 6 months of the last dose of the anti-CD20 antibody; and,
wherein the VIB551 is administered intravenously at a dose of 300 mg every 6 months.

63. The method of claim 61 or claim 62, wherein the NMOSD-related attacks suffered by the patient comprise any one or more of an optic neuritis, a myelitis, or a brainstem attack.

64. The method of claim 63, wherein the NMOSD-related attacks suffered by the patient are clinically asymptomatic.

65. The method of any one of claims 36-64, wherein the patient receives at least one initial dose of VIB551.

66. The method of claim 65, wherein the VIB551 is administered intravenously with a first initial dose of 300 mg, a second initial dose of 300 mg two weeks after the first initial dose, and subsequent doses of 300 mg every 6 months following the first initial dose.

67. The method of claim 66, wherein oral corticosteroids are co-administered to the patient with the initial 300 mg VIB551 dose.

68. The method of claim 67, wherein the oral corticosteroids are administered daily for at least 2 weeks.

69. The method of any one of claims 36-68, wherein the anti-CD20 antibody is rituximab.

70. The method of any one of claims 1-69, wherein the VIB551 comprises a heavy chain variable region (VH) comprising the amino acid of SEQ ID NO:1 and a light chain variable region (VL) comprising the amino acid of SEQ ID NO: 2.

71. A method of treating NMOSD, the method comprising:
administering an anti-CD19 antibody to a patient in need of treatment for NMOSD,
wherein the patient had been previously treated with an anti-CD20 antibody, wherein the patient had an NMOSD attack while being treated with the anti-CD20 antibody.

72. A method of treating NMOSD, the method comprising:
administering an anti-CD19 antibody to a patient in need of treatment for NMOSD,
wherein the patient had been previously treated with an anti-CD20 antibody, wherein the patient had an NMOSD attack within 6 months of the last dose of the anti-CD20 antibody.

73. A method of treating NMOSD, the method comprising:
administering the anti-CD19 antibody VIB551 to a patient in need of treatment for NMOSD,
wherein the patient is an AQP4-IgG⁺ patient,
wherein the VIB551 is administered intravenously with a first initial dose of 300 mg, a second initial dose of 300 mg two weeks after the first initial dose, and subsequent doses of 300 mg every 6 months following the first initial dose,
wherein patient had been previously treated with an anti-CD20 antibody, and wherein the patient had an NMOSD attack while being treated with the anti-CD20 antibody.

74. A method of treating NMOSD, the method comprising:
administering the anti-CD19 antibody VIB551 to a patient in need of treatment for NMOSD,
wherein the patient is an AQP4-IgG⁺ patient,
wherein the VIB551 is administered intravenously with a first initial dose of 300 mg, a second initial dose of 300 mg two weeks after the first initial dose, and subsequent doses of 300 mg every 6 months following the first initial dose,
wherein the patient had been previously treated with an anti-CD20 antibody; and, wherein the patient had an NMOSD attack within 6 months of the last dose of the anti-CD20 antibody.

75. The method of any one of claims 1-74, further comprising, prior to the administering:
identifying the patient as having previously been treated with the anti-CD20 antibody;
determining that the patient:
(i) suffered at least one NMOSD attack while being treated with the anti-CD20 antibody; or
(ii) had suffered at least one NMOSD attack within 6 months of the last dose of the anti-CD20 antibody; and
selecting the patient for the administering the anti-CD19 antibody as a result of the determining (i) or (ii).
